(19) United States
(12) Patent Application Publication
(10) Pub. No.: US 2008/0154144 A1
(43) Pub. Date: Jun. 26, 2008

(54) SYSTEMS AND METHODS FOR CARDIAC CONTRACTILITY ANALYSIS

(76) Inventors: Kamil Unver, Santa Monica, CA (US); Damon J. Coffman, Portland, OR (US); Tat-Jin Teo, Sunnyvale, CA (US); Arvind Thiagarajan, Chennai (IN)

Correspondence Address: KANG LIM, 3494 CAMINO TASSAJARA ROAD #436, DANVILLE, CA, 94506, US

(21) Appl. No.: 11/955,267

(22) Filed: Dec. 12, 2007

Related U.S. Application Data
(63) Continuation-in-part of application No. 11/762,930, filed on Jun. 14, 2007.
(60) Provisional application No. 60/821,752, filed on Aug. 8, 2006.

(51) Int. Cl. A61B 5/02

(52) U.S. Cl. 600/528

(57) ABSTRACT

A method and system of cardiac contractility analysis is provided. Cardiac contractility may include indices such as ejection fraction (EF) and rate of change in pressure (dP/dt) in a heart. Heart sounds may be measured and calibrated by attenuation. Likewise, a first acoustic peak in the first heart sound (S1), and a second acoustic peak of the second heart sound (S2) may be identified. The first heart sound (S1) may be calibrated by the second heart sound (S2). Amplitudes of calibrated heart sounds may be correlated to cardiac contractility. Electrical activity and acoustics of the heart are measured. The pre-ejection period of the cardiac cycle may be calculated. The left ventricular ejection time of the cardiac cycle may likewise be calculated. Then a ratio of pre-ejection period over left ventricular ejection time may be calculated and correlated to cardiac contractility. Pressure on the acoustic sensor may be used to calibrate acoustic data.

### CROSS REFERENCE TO RELATED APPLICATIONS

This is a continuation-in-part of co-pending United States Application Attorney Docket Number HD-0701, application Ser. No. 11/762,930, filed on Jun. 14, 2007, entitled “Systems and Methods for Calibration of Heart Sounds”, which is hereby fully incorporated by reference.

### BACKGROUND OF THE INVENTION

This invention relates generally to medical electronic devices for analysis of cardiac efficiency as measured by cardiac contractility. Cardiac contractility may include rate of pressure change in a heart, and ejection fraction of the heart. More particularly, this invention relates to a method for improving medical heart diagnosis through noninvasive procedures, by determining the ability of the heart to generate the necessary pressure in a timely manner (dP/dt).

The heart has four chambers—two upper chambers (called atria) and two lower chambers (ventricles). The heart has valves that temporarily close to permit blood flow in only one direction. The valves are located between the atria and ventricles, and between the ventricles and the major vessels from the heart. In healthy adults, there are two normal heart sounds: a first heart sound (S1) and second heart sound (S2). The first heart sound is produced by the closure of the Atrioventricular (AV) valves and the second heart sound is produced by semilunar valves closure.

Moreover, in addition to these normal sounds, a variety of other sounds may be present, including heart murmurs and adventitious sounds, or clicks. Murmurs are blowing, whooshing, or rasping sounds produced by turbulent blood flow through the heart valves or near the heart. Murmurs can happen when a valve does not close tightly, such as with mitral regurgitation which is the backflow of blood through the mitral valve, or when the blood is flowing through a narrowed opening or a stiff valve, such as with aortic stenosis. A murmur does not necessarily indicate a disease or disorder, and not all heart disorders cause murmurs.

Murmurs may be physiological (benign) or pathological (abnormal). Different murmurs are audible in different parts of the cardiac cycle, depending on the cause and grade of the murmur. Significant murmurs can be caused by: chronic or acute mitral regurgitation, aortic regurgitation, aortic stenosis, tricuspid stenosis, tricuspid regurgitation, pulmonary stenosis and pulmonary regurgitation

The first heart tone, or S1, is caused by the sudden block of reverse blood flow due to closure of the mitral and tricuspid atrioventricular valves at the beginning of ventricular contraction, or systole.

The second heart tone, or S2, marks the beginning of diastole, the heart's relaxation phase, when the ventricles fill with blood. The second heart sound is caused by the sudden block of reversing blood flow due to closure of the aortic valve and pulmonary valve. In children and teenagers, S2 may be more pronounced. Right ventricular ejection time is slightly longer than left ventricular ejection time.

A third heart sound, or S3, may be heard at the apex. This sound usually occurs approximately 0.15 seconds after the second heart sound. The third heart sound is a low pitched soft blowing sound. It may be caused by congestive heart failure, fluid overload, cardiomyopathy, or ventricular septal defect, but can also occur normally in young persons. The third heart sound usually occurs whenever there is a rapid heart rate, such as over 100 beats per minute (bpm). The third heart sound is caused by vibration of the ventricular walls, resulting from the first rapid filling. However, it may also be found in young persons, pregnant women or people with anemia with no underlying pathology.

The fourth heart sound S4 occurs during the second phase of ventricular filling: when the atriums contract just before S1. As with S3, the fourth heart sound is thought to be caused by the vibration of valves, supporting structures, and the ventricular walls. An abnormal S4 is heard in people with conditions that increase resistance to ventricular filling, such as a weak left ventricle.

Auscultatory sounds have long been the primary inputs to aid in the noninvasive detection of various physiological conditions. For instance the stethoscope is the primary tool used by a clinician to monitor heart sounds to detect and diagnose the condition of a subject's heart. Auscultation itself is extremely limited, thus far, by a number of factors. It is extremely subjective and largely depends on the clinician's expertise in listening to the heart sounds and is compounded by the fact that certain components of the heart sounds are beyond the gamut of the human ear.

By definition, the volume of blood within a ventricle immediately before a contraction is known as the end-diastolic volume. Similarly, the volume of blood left in a ventricle at the end of contraction is end-systolic volume. The difference between end-diastolic and end-systolic volumes is the stroke volume, the volume of blood ejected with each beat. Ejection fraction (EF) is the fraction of the end-diastolic volume that is ejected with each beat; that is, it is stroke volume (SV) divided by end-diastolic volume (EDV).

The term ejection fraction applies to both the right and left ventricles; one can speak equally of the left ventricular ejection fraction (LVEF) and the right ventricular ejection fraction (RVEF). Without a qualifier, the term ejection fraction refers specifically to that of the left ventricle.

In a healthy 70-kg (154-lb) man, the SV is approximately 70 ml and the left ventricular EDV is 120 ml, giving an ejection fraction of 70/120, or 58%. Right ventricular volumes being roughly equal to those of the left ventricle, the ejection fraction of the right ventricle is normally equal to that of the left ventricle within narrow limits.

Damage to the muscle of the heart (myocardium), such as that sustained during myocardial infarction or in cardiomyopathy, impairs the heart's ability to eject blood and therefore reduces ejection fraction. This reduction in the ejection fraction can manifest itself clinically as heart failure.

The maximum ratio of pressure change to time change, or rate of pressure change during ventricular contraction (dP/dt) relates to ejection fraction in that the maximum dP/dt occurs during isovolumetric contraction. This occurs because as the heart walls contract, volume decreases. Blood is then forced out of the ventricular valves along a pressure gradient.

The maximum dP/dt is a very effective indicator of ventricular performance. This is due to the sensitivity this ratio to changes in contractility, yet relative insensitivity to changes in after load, and preload. Also, the ratio of pressure change to time change is not affected by variations in ventricular anatomy and motion anomalies common to patients with congenital heart disease.

Traditionally, dP/dt measurement requires insertion of an intraventricular catheter. Such methods are expensive, uncomfortable, and require incisions and long recovery time. Due to the cost benefits, ease of use, and minimal invasiveness of heart sound measurements, a preferred system of utilizing acoustic measurements to determine dP/dt ratio is desired.

It is therefore apparent that an urgent need exists for an improved auscultatory device capable of noninvasive determination of cardiac contractility as measured through the maximum rate of pressure change within the heart.

### SUMMARY OF THE INVENTION

To achieve the foregoing and in accordance with the present invention, a method and system of cardiac contractility analysis is provided. Cardiac contractility may include the rate of change in pressure in a heart, as well as ejection fraction of the heart. Such a system is useful for a clinician to efficiently and accurately diagnose heart patients.

An embodiment of the method and system of cardiac contractility analysis, by measuring heart sound amplitudes is provided. In this embodiment, heart sounds may be calibrated and correlated to pressure changes in the heart. A transducer may be placed upon a cardiac patient. Likewise a sensor may be oriented on the cardiac patient. The sensor may include a pressure sensor for measuring the pressure of the placement of the sensor on the heart patient's body.

In some embodiments, an audio signal may be generated by the transducer for measurement by the sensor. From this measurement an attenuation signal may be generated. The sensor may also measure heart sounds. These heart sounds may then be calibrated by the attenuation signal, as well as the pressure measurement by the pressure sensor. Finally, the cardiac contractility may be computed by correlation to the amplitudes of the calibrated heart sounds.

In some other embodiments, only the sensor is required. The heart sounds may again be calibrated for pressure of the sensor on the patient's body. The sensor may measure the first and second heart sounds. The first heart sound may be calibrated by the second heart sound. The cardiac contractility may be computed by correlation to this calibrated heart sound.

An alternate embodiment of the method and system of cardiac contractility analysis, by means of looking at timing intervals is also provided. This embodiment may be secondary to, or complimentary to, the cardiac contractility analysis by means of utilizing heart sound amplitudes. This embodiment requires measuring the electrical activity of the heart. This may be done by the electrocardiograph. The electrical activity is used to determine the initiation of the cardiac cycle. Additionally, acoustic data of the heart is collected.

An acoustic peak in the first heart sound (S1) caused by the closure of atrioventricular valves in the heart may be identified. The time interval from the initiation of the electrical cycle to the peak of S1 is defined as the Electromechanical Activation Time (EMAT). Although not strictly equivalent to Pre-Ejection Period (PEP) (normally defined as the time interval from the initiation of the electrical cycle to the opening of the aortic cycle), it can be used analogously except in extreme cases.

By correlation to the pre-ejection period, the cardiac contractility may be determined.

Additionally, the acoustic peak of the second heart sound (S2), caused by the closure of the aortic and pulmonary valves may be identified. By measuring the time interval between the peak of S1 and the peak of S2, the left ventricular ejection time (LVET) may be measured. It should be noted, however, that the strict definition of LVET is defined as the time interval during which the aortic valve is open. However, this method provides a close analogy which can be used in all but the most extreme cases.

Then a ratio of pre-ejection period over left ventricular ejection time may be calculated. By correlation to this ratio the cardiac contractility may also be generated.

In some embodiments, pressure data of the chest pad used to collect the acoustic data from the heart may be collected. This pressure data may be used to calibrate the acoustic data.

Likewise, an acoustic attenuation signal may be generated and received in order to create an attenuation matrix. The attenuation matrix may also be used to calibrate the collected acoustic data.

Note that the various features of the present invention described above may be practiced alone or in combination. These and other features of the present invention will be described in more detail below in the detailed description of the invention and in conjunction with the following figures.

### BRIEF DESCRIPTION OF THE DRAWINGS

In order that the present invention may be more clearly ascertained, one or more embodiments will now be described, by way of example, with reference to the accompanying drawings, in which:

FIG. 1A illustrates an exemplary pair of transducing and sensing positions for measuring acoustic attenuation of a thoracic region in accordance with the present invention;

FIG. 1B illustrates an exemplary single location echo method for measuring acoustic attenuation of a thoracic region in accordance with the present invention;

FIG. 2 shows exemplary frontal ECG sensing positions located on the thoracic region;

FIG. 3A shows a front view illustrating one embodiment of a chest-patch which combines an ECG sensor with an acoustic transducer for the auscultation device of the present invention;

FIG. 3B shows a side view illustrating one embodiment of a chest-patch which combines an ECG sensor with an acoustic transducer for the auscultation device of the present invention;

FIG. 4A shows a front view illustrating another embodiment of a rectangular chest-patch which combines an ECG sensor with an acoustic transducer for the auscultation device of the present invention;

FIG. 4B shows a side view illustrating another embodiment of a rectangular chest-patch which combines an ECG sensor with an acoustic transducer for the auscultation device of the present invention;

FIG. 5 shows a side view illustrating one exemplary chest-piece which combine an acoustic transducer with an acoustic sensor for the auscultation device of the present invention;

FIG. 6 shows a side view illustrating a second exemplary chest-piece which combine an acoustic transducer with an acoustic sensor for the auscultation device of the present invention;

FIG. 7 shows a bottom view illustrating a third exemplary chest-piece which combines an acoustic transducer with an acoustic sensor in separate acoustic cavities for the auscultation device of the present invention;

FIG. 8A is a bottom view illustrating yet another chest-pad which includes a triplet of Acoustic Sensors in accordance with an embodiment of the present invention;

FIG. 8B is a bottom view illustrating yet another chest-pad which includes a quintuplet of Acoustic Sensors in accordance with an embodiment of the present invention;

FIG. 8C is a bottom view illustrating yet another chest-pad which includes a sextet of Acoustic Sensors in accordance with an embodiment of the present invention;

FIG. 9 shows an exemplary diagram of pressure, timing, blood volume and signals associated in a typical cardiac cycle;

FIG. 10A shows a functional block diagram of one embodiment of the auscultatory device in accordance with an embodiment of the present invention;

FIG. 10B shows a functional block diagram of another embodiment of the auscultatory device in accordance with an embodiment of the present invention;

FIG. 10C shows a functional block diagram of yet another embodiment of the auscultatory device in accordance with an embodiment of the present invention;

FIG. 10D shows a functional block diagram of yet another embodiment of the auscultatory device in accordance with an embodiment of the present invention;

FIG. 11 shows an illustration of a functional block diagram for the analyzer in accordance with an embodiment of the present invention;

FIG. 12 provides a detailed block diagram illustrating heart sound signal conditioner in accordance with an embodiment of the present invention;

FIG. 13 shows an exemplary process for self calibration of heart signals utilizing an embodiment of the auscultatory device;

FIG. 14 shows an exemplary process for signal conditioning of heart signals utilizing an embodiment of the auscultatory device;

FIG. 15 shows an exemplary process for generating the attenuation matrix utilizing an embodiment of the auscultatory device;

FIG. 16 shows an exemplary process for pulsed echo utilizing an embodiment of the auscultatory device;

FIG. 17 shows an exemplary process for motion detection in pulsed echo utilizing an embodiment of the auscultatory device;

FIG. 18 shows an exemplary process for structure speed detection in pulsed echo utilizing an embodiment of the auscultatory device;

FIG. 19 shows an exemplary process for using the auscultatory device to determine cardiac contractility in accordance with an embodiment of the present invention;

FIG. 20 shows an exemplary process for signal processing for cardiac contractility determination in accordance with an embodiment of the present invention;

FIG. 21A shows an exemplary illustration of ECG and sound wave measurements for usage by cardiac contractility analysis;

FIG. 21B shows an exemplary illustration of sound wave measurements for usage by cardiac contractility analysis;

FIG. 22 shows an exemplary illustration of isolated ECG and sound wave measurement for usage by cardiac contractility analysis;

FIG. 23A shows an exemplary illustration of ECG and sound wave measurements when attenuation signal is applied for usage by cardiac contractility analysis;

FIG. 23B shows an exemplary illustration of sound wave measurements when attenuation signal is applied for usage by cardiac contractility analysis;

FIG. 24 shows an exemplary illustration of filtered measured transduction signals for cardiac contractility analysis;

FIG. 25A shows an exemplary illustration of pre-filtered measured heart sound signals for cardiac contractility analysis;

FIG. 25B shows an exemplary illustration of post-filtered measured heart sound signals for cardiac contractility analysis;

FIG. 26 shows an exemplary illustration of measured heart sound signals before and after de-noising for cardiac contractility analysis;

FIG. 27A shows a front view illustrating one embodiment of a chest-patch which combines an ECG sensor with an acoustic transducer and a pressure sensor for the auscultation device of the present invention;

FIG. 27B shows a side view illustrating one embodiment of a chest-patch which combines an ECG sensor with an acoustic transducer and a pressure sensor for the auscultation device of the present invention;

FIG. 28 shows a front view illustrating another embodiment of a chest-patch which combines an ECG sensor with an acoustic transducer and a pressure sensor for the auscultation device of the present invention;

FIG. 29 shows a front view illustrating yet another embodiment of a chest-patch which combines an ECG sensor with an acoustic transducer and a pressure sensor for the auscultation device of the present invention;

FIG. 30 shows a functional block diagram of another embodiment of the auscultatory device in accordance with an embodiment of the present invention;

FIG. 31 shows a logical block diagram illustrating an embodiment of a signal processor in accordance with an embodiment of the present invention;

FIG. 32 shows a logical block diagram illustrating an embodiment of a rate of pressure change generator in accordance with an embodiment of the present invention;

FIG. 33 shows a logical block diagram illustrating an embodiment of a timing analyzer in accordance with an embodiment of the present invention;

FIG. 34A shows an exemplary process for determining the rate of pressure change in a heart in accordance with an embodiment of the auscultatory device;

FIG. 34B shows another exemplary process for determining the rate of pressure change in a heart in accordance with an embodiment of the auscultatory device;

FIG. 35 shows an exemplary process for conditioning data signals in accordance with an embodiment of the auscultatory device;

FIG. 36 shows an exemplary process for selecting a S1 acoustic peak in accordance with an embodiment of the auscultatory device;

FIG. 37 shows an exemplary process for selecting a S2 acoustic peak in accordance with an embodiment of the auscultatory device;

FIG. 38 shows an exemplary process for calculating pre-ejection period in accordance with an embodiment of the auscultatory device;

FIG. 39 shows an embodiment of an exemplary process for calculating left ventricular ejection time in accordance with an embodiment of the auscultatory device;

FIG. 40 shows an exemplary process for generating the rate of pressure change in a heart in accordance with an embodiment of the auscultatory device; and

FIG. 41 shows an exemplary illustration of a sound wave measurement for usage in determining the rate of pressure change in a heart.

### DETAILED DESCRIPTION OF THE INVENTION

The present invention will now be described in detail with reference to several embodiments thereof as illustrated in the accompanying drawings. In the following description, numerous specific details are set forth in order to provide a thorough understanding of the present invention. It will be apparent, however, to one skilled in the art, that the present invention may be practiced without some or all of these specific details. In other instances, well known process steps and/or structures have not been described in detail in order to not unnecessarily obscure the present invention. The features and advantages of the present invention may be better understood with reference to the drawings and discussions that follow.

Systems and methods for cardiac contractility analysis are provided. Cardiac contractility may include rate of pressure change in a heart, and ejection fraction of the heart. The present invention utilizes noninvasive measurements including phonocardiograph and, in some embodiments, electrocardiogram (ECG) in order to generate the rate of pressure change (dP/dt) or ejection fraction (EF) in a heart. These measures are useful in the diagnosis of a heart failure patient.

In some embodiments, heart sounds, as measured by an acoustic sensor, may be calibrated by a generated acoustic attenuation signal. An audio signal may be generated by a transducer for measurement by a sensor. From this measurement the attenuation signal may be generated. The sensor may also measure heart sounds. The rate of change in pressure (dP/dt) in the heart may be computed by correlation to the amplitudes of the calibrated heart sounds.

In some other embodiments, only a sensor is required. The sensor may measure the first and second heart sounds. The first heart sound may be calibrated by the second heart sound. The rate of change in pressure (dP/dt) in the heart may be computed by correlation to this calibrated heart sound.

In some alternate embodiments, an electrocardiogram may be used to determine the initiation of a cardiac cycle. Subsequently, an auscultatory device may be utilized to determine Pre-ejection Period (PEP) and Left Ventricular Ejection Time (LVET) in order to correlate to the rate of pressure change (dP/dt) in a heart. Such timing based embodiments may supplement either of the forgoing embodiments of pressure change measurement by utilizing calibrated heart sounds. Of course, any of the disclosed embodiments of measuring the rate of pressure change (dP/dt) in a heart are intended as being capable of being performed alone or in combination.

In the foregoing embodiments, pressure sensors may be utilized to calibrate the audio data from the auscultatory device. The pressure sensor may measure the sensor placement on the patient's body. Signal attenuation may additionally be utilized in some embodiments for calibration.

In some alternate embodiments, attenuation systems are not available or practical. It should be noted that the disclosed invention is capable of performing with non attenuation calibrated data.

The present invention will be disclosed as a series of electro-mechanical auscultation devices enabled to perceive the required signals and calculate the rate of pressure change in the heart.

Particular subheadings are included to provide guidance and organization to the disclosure. These sub headings are not intended to suggest or impose limitations upon the disclosed invention.

### Auscultation Devices

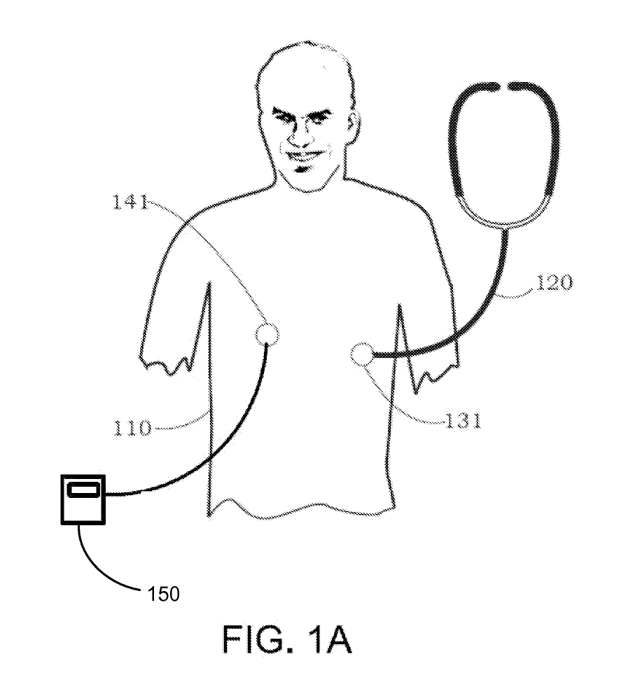

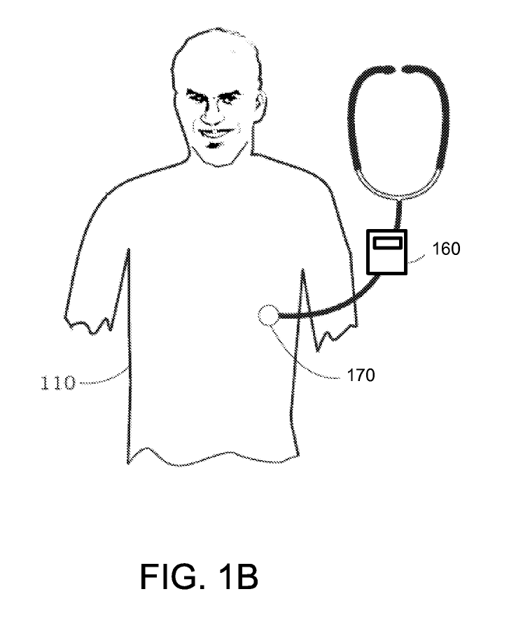

To facilitate discussion, FIG. 1A shows an exemplary pair of transducing and sensing positions for measuring acoustic attenuation of the thoracic region 110 using an auscultation device, e.g., an Electronic Stethoscope 120, of the present invention. Such an auscultation device includes an Acoustic Transducer 150 coupled to transducing position 141, and an acoustic sensor or stethoscope 120, coupled to sensing location 131. Additional pairs of transducing and sensing positions may be used to generate an acoustic attenuation map of thoracic region 110.

A suitable acoustic signal of known amplitude and frequency, e.g. a sine wave, may be provided by the Acoustic Transducer 150 at Transducing Location 141. Since one primary object of the invention is to measure and compensate for the acoustic attenuation of S1, S2, S3, S4 heart sounds and heart murmurs as these heart sounds travel from the heart to the acoustic sensor of Stethoscope 120, the acoustic signal may include a frequency range of about 50 Hz to 300 Hz. Depending on the implementation, this acoustic signal may include a series of stepped frequencies, a swept range of frequencies and/or multi-frequency signals.

In alternate embodiments, the acoustic signal from the transducer may have an acoustic frequency of 1 MHz and higher. Such embodiments enable the transducer signal to be filtered from the heart sounds by the Stethoscope 120. Additionally, such frequency range may provide directional information through Doppler analysis that would not be ascertainable at lower frequency transducer signal.

Additionally, in some embodiments the transducer signal may be pulsed as to minimize interference with the Stethoscope 120 microphone. Such a pulsed transducer signal, or echo pulse, may be relatively short, e.g., on the order of microseconds up to tens of microseconds.

The attenuated signal received at Sensing Location 131 is digitized, and may be analyzed in the frequency and/or time domain. For example, comparison of the digitized attenuated signal against the initial transduced signal allows for the computation of the degree of attenuation between Location 141 and Location 131. The computed degree of attenuation may be a single constant of volume attenuation or a multi-value measurement of attenuation of volume at one or more frequencies. This measurement of attenuation may also include time variant measurements as a function of frequency. Other standard signal processing techniques known to one skilled in the arts may also be used to compute attenuation.

By taking measurements from suitable pairs of transducing and sensing locations distributed over the area of interest, a matrix of the attenuation may be compiled. Subsequently, this attenuation matrix may be used to calibrate heart sounds to compensate for acoustic attenuation caused by the intervening tissues and fluids between the heart and the sensor, thereby increasing the accuracy of the diagnosis of the various heart sounds and murmurs.

FIG. 1B shows an exemplary diagram of transducer placement for pulse echo devices. In such embodiments, the transducer and sensor may be located within the Echo Auscultation Device 160. Thus, in these embodiments, the Sensing Location 131 and Transducing Position 141 may be adjacent to one another, or may be the same Common Location 170.

The Echo Auscultation Device 160 provides the acoustic signal and subsequently senses the return echo, at the same Common Location 170 on the patient. Thus comfort and simplicity of the system is improved since there is only one pad needed.

As noted above, a suitable acoustic signal of known amplitude and frequency, e.g., a sine wave, may be provided by the acoustic transducer portion of the Echo Auscultation Device 160 at the Common Location 170. Again, the acoustic signal may include a frequency range of about 50 Hz to 300 Hz or may have an acoustic frequency of 1 MHz and higher. Depending on the implementation, this acoustic signal may include a series of stepped frequencies, a swept range of frequencies and/or multi-frequency signals.

Additionally, in some embodiments the transducer signal may be pulsed as to minimize interference from acoustic signal generation and acoustic measurements. Such a pulsed transducer signal, or echo pulse, may be relatively short, e.g., on the order of tens of microseconds.

The pulse echo is received at the Common Location 170, where it is digitized, and may be analyzed in the frequency and/or time domain. Other standard signal processing techniques known to one skilled in the arts may also be used to compute analysis. Echo patterns may be compiled within an attenuation matrix, which may be used to calibrate heart sounds to compensate for acoustic attenuation caused by the intervening tissues and fluids between the heart and the sensor, thereby increasing the accuracy of the diagnosis of the various heart sounds and murmurs.

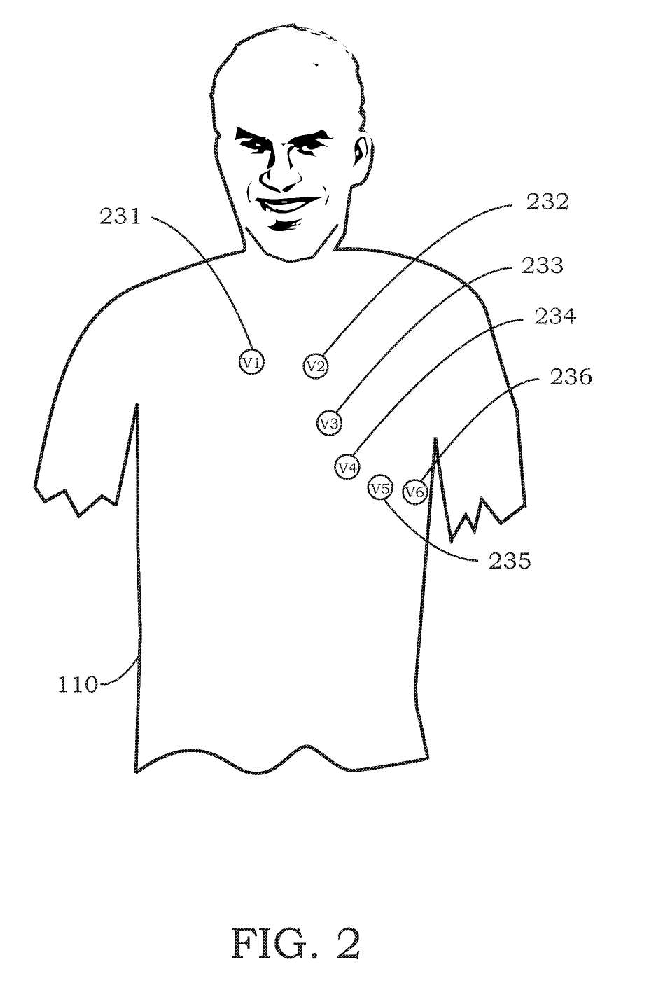

FIG. 2 shows a selection of suitable auscultation sensing locations. These locations include aortic, pulmonary, mitral, tricuspid and apex locations. Other exemplary sensing locations include typical ECG sensing locations 231, 232, 233, 234, 235, 236 corresponding to anterior thoracic ECG positions V1, V2, V3, V4, V5 and V6 may also be used as shown in FIG. 2. Additional thoracic ECG sensing locations such as posterior ECG positions V7, V8 and V9 (not shown) may also be used. Other auscultation locations known to one skilled in the cardiac diagnostic arts may also be used.

In some embodiments, the method for measuring heart sounds is performed to identify motion within the chest cavity. When the sensory location is fixed on the patient's torso, the received acoustic signals are processed for structures and fluids along the acoustic path.

A “brightness line” image may be generated from the received acoustic signals as to provide a representation for the structures along the acoustic path. By maintaining a fixed acoustic path, and repeatedly sensing the structures, motion may be identified and tracked. A heart valve is in motion with respect to the patient's chest wall, thus the distance of the valve to the chest wall may be deduced. Such a deduction may accurately be used to enable the calibration of the heart sound of that particular patient to his chest size or attenuation characteristics (the amount of subcutaneous fat, for example).

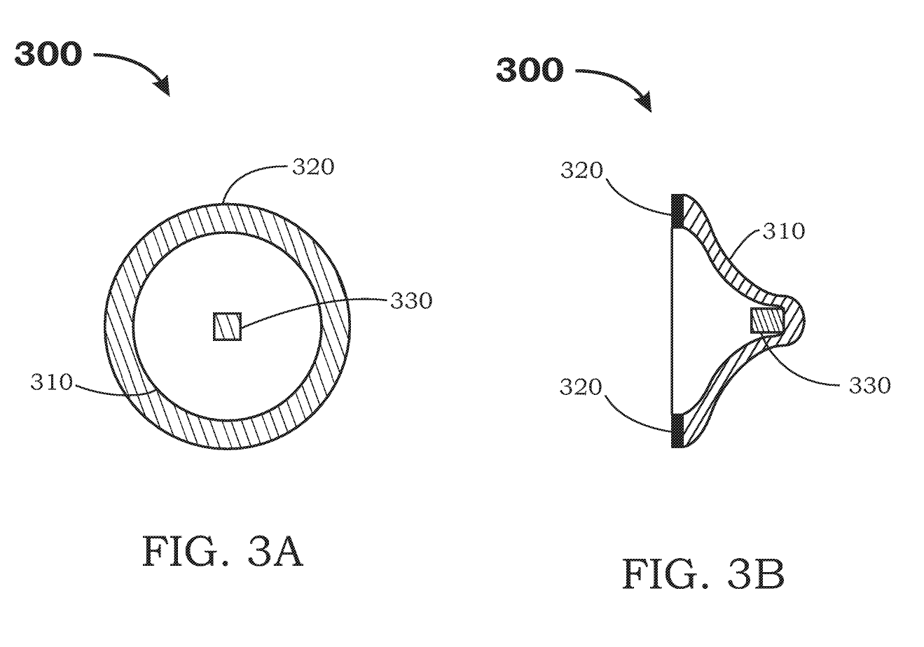

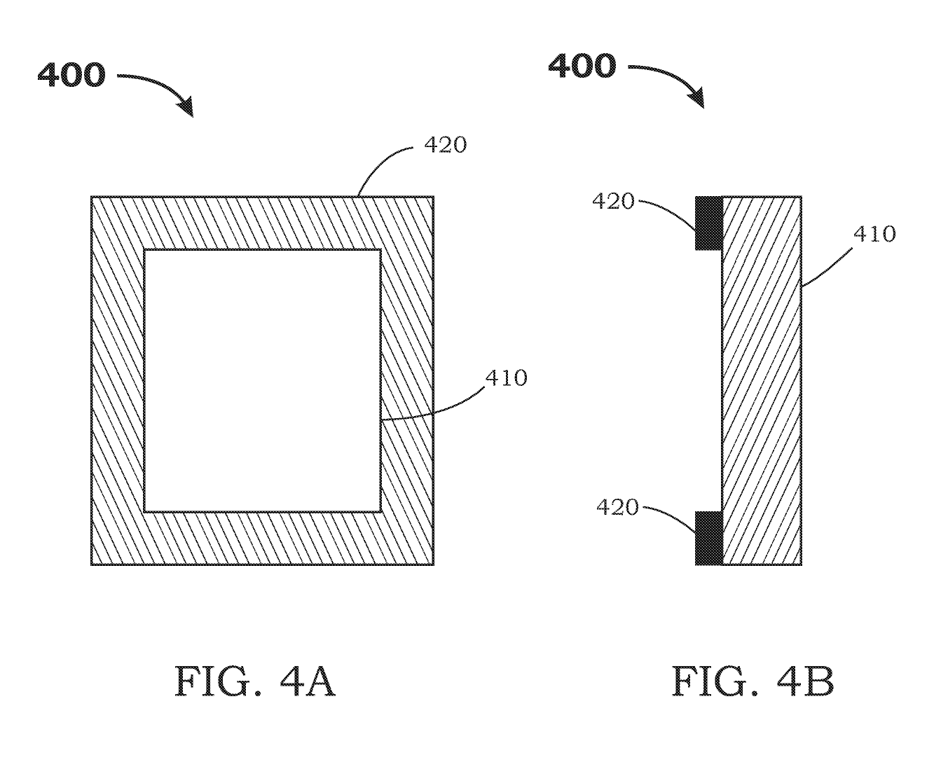

FIGS. 3A and 3B are front and side views illustrating one embodiment 300 of the present invention which combines an ECG sensor 320 and an acoustic transducer 330 housed in a bell-shaped body 310. In this embodiment, ECG sensor 320 is a conductive ring allowing ECG electrical signal transmission from the base of body 310. The bell-shaped body 310 focuses the acoustic signal generated by acoustic transducer 330, e.g., a miniature speaker, located at the top of body 310. ECG sensor 320 may include a sealing membrane to ensure both electrical conduction and mechanical air seal for superior acoustic transmission. Sealing may also be accomplished by an ECG gel in combination with or in place of a sealing membrane. Bell-shaped body 310 may be filled with air or fluid to facilitate acoustic transmission.

The Acoustic Transducer 330 may, in some embodiments, be a traditional membrane and magnet speaker. Alternatively, Acoustic Transducer 330 may be a piezo transducer. Of course additional transducers may be utilized as is known by those skilled in the art.

A piezo Acoustic Transducer 330 may be capable of producing an acoustic signal, as well as sensing acoustic waves. Thus the Acoustic Transducer 330, in some embodiments where piezo or similar designs are utilized, may both supply the acoustic signal as well as provide sensory reception. Such a transducer may be utilized in the Pulse Echo Unit 170 of FIG. 1B. In these embodiments the Acoustic Transducer 330 provides a pulse of acoustic signal. During pulse generation the Acoustic Transducer 330 is unable to provide sensory, thus the length of pulse may be limited to a practical duration. In some embodiments, pulse duration of 10-30 microseconds is sufficient. The average cardiac cycle is on the magnitude of a full second, thus the pulse is a relatively short time for the Acoustic Transducer 330 to be unable to sense acoustic signals. Moreover, by interleaving the pulse and heart sounds over the cardiac signal, data loss may be mitigated.

In some alternate embodiments, the Acoustic Transducer 330 may be designed to only generate acoustic signals. Such an embodiment may be utilized in the separated Acoustic Transducer 150 and Stethoscope 120 design as illustrated in FIG. 1A. In these embodiments, the Acoustic Transducer 330 may provide pulse acoustic signals, constant acoustic signals or a combination thereof.

FIGS. 4A and 4B are front and side views illustrating one embodiment 400 of the present invention which combines an ECG sensor 420 and an acoustic transducer in a flat housing 410 which may be square-shaped as shown, or may be another suitable shape such as rectangular, polygonal, or oval. Acoustic transducer may be a piezoelectric element coupled to the base of housing 410, or may include additional acoustic generator designs, such as traditional speakers.

Again, the embodiment seen generally at 400 may include both acoustic generation and sensory, or may be limited to generation only, dependent on whether an echo type design, or a separated transducer and sensor design is required, as seen in FIGS. 1B and 1A, respectively.

ECG sensor 420 may include a sealing membrane to ensure both electrical conduction and mechanical air seal for superior acoustic transmission. Sealing may also be accomplished by an ECG gel in combination with or in place of a sealing membrane.

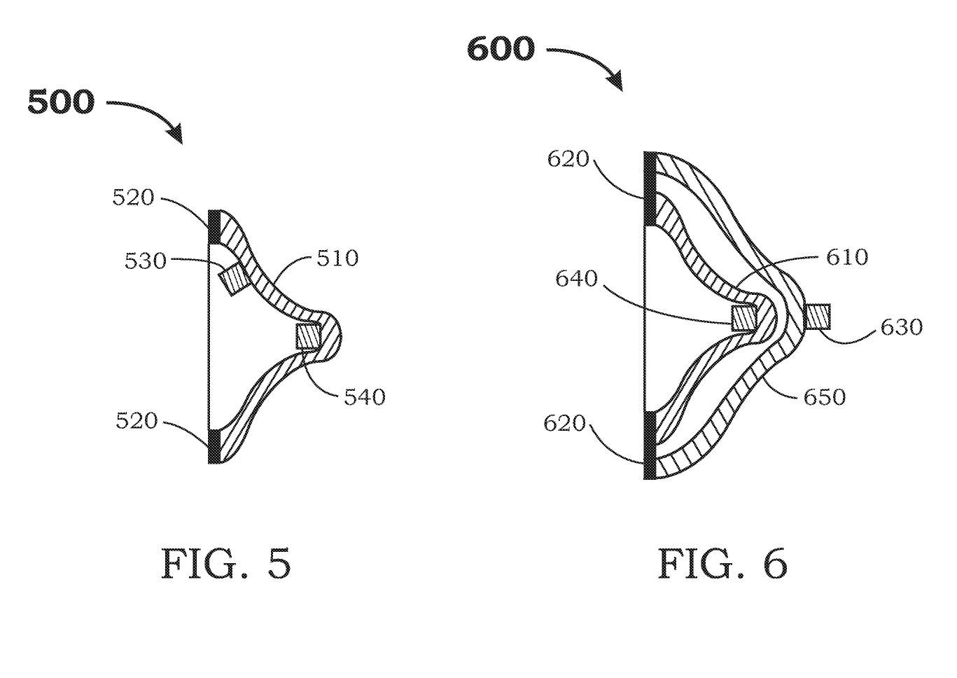

FIG. 5 is a side view illustrating one embodiment of a chest-piece 500 which combines an acoustic transducer 530 with an acoustic sensor 540 in a bell shaped housing 510, the chest-piece 500 useful with the auscultation device of the present invention. Such a device may be utilized in an echo type method as illustrated in FIG. 1B. Acoustic transducer 530 and an acoustic sensor 540 may be piezos; however traditional microphone and speaker arrangements may also be utilized.

The acoustic sensor 540 may be sensitive to sound frequencies between 10 Hz to 500 Hz as well as frequencies generated by the acoustic transducer 530. Thus the acoustic sensor 540 may provide auscultation as well as attenuation measurement for calibration. Alternatively, in some embodiments, the acoustic transducer 530 generates sound waves in the MHz range, and it may be more desirable for the acoustic sensor 540 to be comprised of multiple sensors to cover the range of physiological and generated sound waves. Thus one benefit of a separate acoustic sensor 540 may be a more sensitive sensory capability across a greater frequency range.

An additional benefit of separate acoustic transducer 530 and acoustic sensor 540 is the elimination of the sensory blindness that occurs during generation when a single transducer is utilized. As such, a chest-piece as illustrated generally at 500 may provide continuous, as well as pulse acoustic attenuation.

ECG sensor 520 may include a sealing membrane to ensure both electrical conduction and mechanical air seal for superior acoustic transmission. Sealing may also be accomplished by an ECG gel in combination with or in place of a sealing membrane.

FIG. 6 is a side view of another exemplary chest-piece 600 which includes an acoustic transducer 630 located in an outer annulus 650 combined with an acoustic sensor 640 located on an inner sensing bell 610, the chest-piece 600 useful with the auscultation device of the present invention.

The chest piece depicted generally at 600 provides the same functionalities as the one shown at FIG. 5; however, by separating the acoustic transducer 630 from the acoustic sensor 640 within separate bells, there may be a reduction in interference from the acoustic transducer 630 signal and the acoustics received by the acoustic sensor 640. Again the acoustic sensor 640 may be a sensory array, enabled to sense across a wide range of sound frequencies.

ECG sensor 620 may include a sealing membrane to ensure both electrical conduction and mechanical air seal for superior acoustic transmission. Sealing may also be accomplished by an ECG gel in combination with or in place of a sealing membrane.

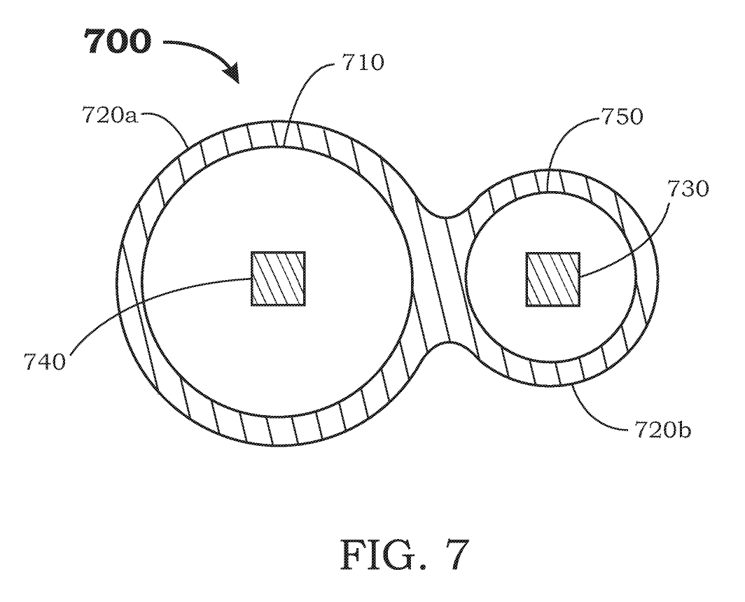

FIG. 7 is a bottom view illustrating yet another chest-piece 700 which includes an acoustic sensor 740 located in a sensing cavity 710 combined with an acoustic transducer 730 located in an attached auxiliary cavity 750. Cavities 710, 750 function as independent acoustic chambers to minimize cross-interference between transducer 730 and sensor 740. Optional sealing membrane 720a, 720b may be added to improve the acoustic properties of cavities 710, 750, respectively.

Although not illustrated, the chest-piece 700 may include an ECG sensor, which may include a sealing membrane to ensure both electrical conduction and mechanical air seal for superior acoustic transmission. Sealing may also be accomplished by an ECG gel in combination with or in place of a sealing membrane.

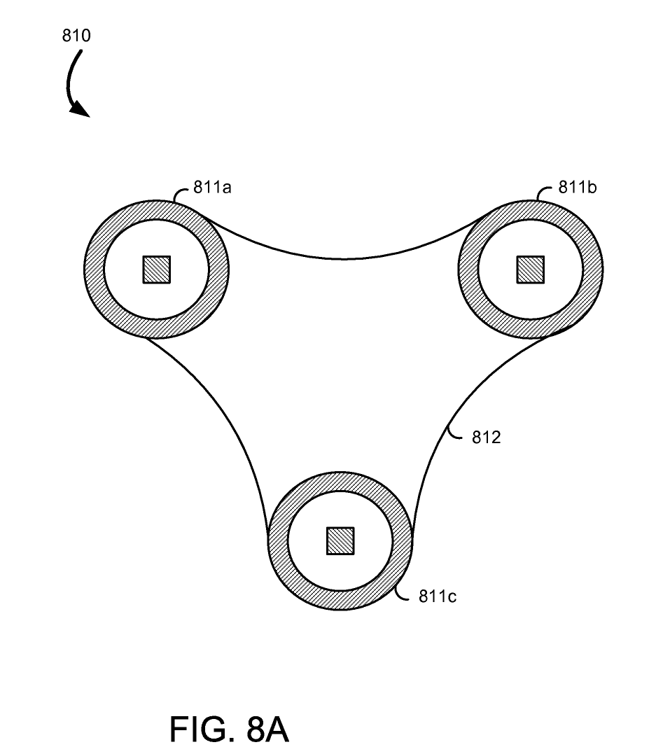

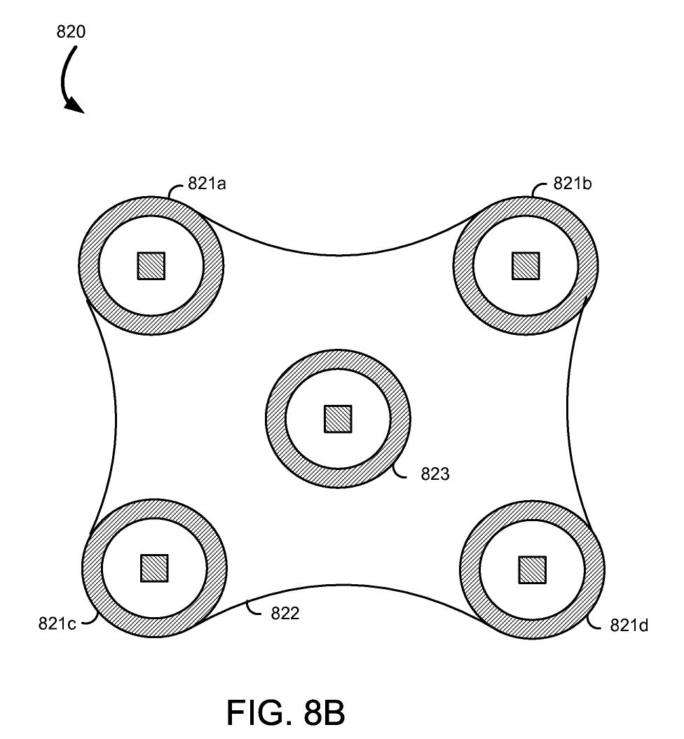

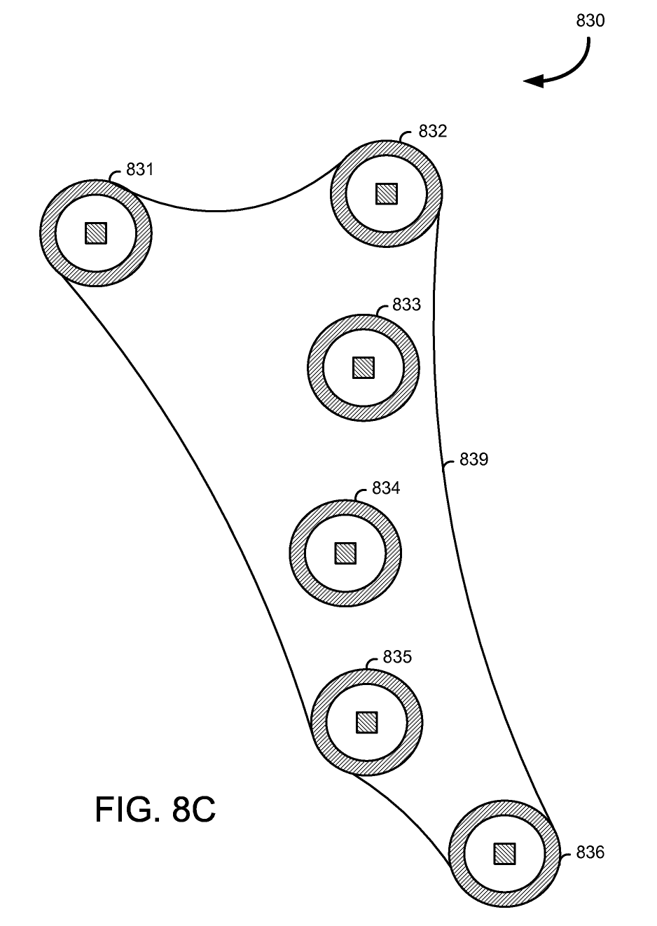

FIG. 8A is a bottom view illustrating yet another chest-pad 810 which includes a triplet of Acoustic Sensors labeled 811a, 811b and 811c, respectively. Acoustic Sensors 811a, 811b and 811c may be interconnected by a Webbing 812.

Webbing 812 may, in some embodiments, be a cloth mesh or plastic. Alternatively, Webbing 812 may be rigid in nature and include metal or plastics. In some embodiments, Webbing 812 may be connector rods of any suitable material. Webbing 812 functions to maintain the relative positions of the Acoustic Sensors 811a, 811b and 811c to one another.

Acoustic Sensors 811a, 811b and 811c may be arranged in an isosceles triangular fashion. Alternate orientations may additionally be utilized as is desired. In some embodiments, Acoustic Sensors 811a, 811b and 811c may be bell shaped sensor pads, with a microphone or piezo sensor in the vertex of the bell. Additionally, Acoustic Sensors 811a, 811b and 811c may include ECG functionality.

Acoustic Sensors 811a, 811b and 811c may, in some embodiments, additionally provide an active signal through a transducer. In other embodiments, a separate transducer may be utilized to generate the active acoustic signals.

Moreover, perceived signals by the Acoustic Sensors 811a, 811b and 811c may enable depth and location triangulation for internal structures when utilizing echo signals.

In some embodiments, the Chest Pad 810 may be designed in variant sizing for separate body sizes and types. In some embodiments, the Webbing 812 may be elastic as to increase wearer comfort and enable a singular device to be utilized by a wide gamut of individuals.

FIG. 8B is a bottom view illustrating yet another chest-pad 820 which includes a quintuplet of Acoustic Sensors labeled 821a, 821b, 821c, 821d and 823, respectively. Acoustic Sensors 821a, 821b, 821c, 821d and 823 may be interconnected by a Webbing 822. In the present design, Acoustic Sensors 821a, 821b, 821c and 821d are oriented in a square geometry around a central Acoustic Sensor 823. Alternate orientations may additionally be utilized as is desired. The central Acoustic Sensor 823 may, in some embodiments, provide a transducer. Additional Acoustic Sensors 821a, 821b, 821c, 821d and 823 may, in some embodiments, additionally provide an active signal through a transducer. In other embodiments, a separate transducer may be utilized to generate the active acoustic signals.

As previously discussed, Webbing 822 may, in some embodiments, be a cloth mesh or plastic. Alternatively, Webbing 822 may be rigid in nature and include metal or plastics. In some embodiments, Webbing 822 may be connector rods of any suitable material. Webbing 822 functions to maintain the relative positions of the Acoustic Sensors 821a, 821b, 821c, 821d and 823 to one another.

In some embodiments, Acoustic Sensors 821a, 821b, 821c, 821d and 823 may be bell shaped sensor pads, with a microphone or piezo sensor in the vertex of the bell. Additionally, Acoustic Sensors 821a, 821b, 821c, 821d and 823 may include ECG functionality.

Moreover, perceived signals by the Acoustic Sensors 821a, 821b, 821c, 821d and 823 may enable depth and location triangulation for internal structures when utilizing echo signals.

As previously discussed, in some embodiments, the Chest Pad 820 may be designed in variant sizing for separate body sizes and types. In some embodiments, the Webbing 822 may be elastic as to increase wearer comfort and enable a singular device to be utilized by a wide gamut of individuals.

FIG. 8C is a bottom view illustrating yet another chest-pad 830 which includes a sextet of Acoustic Sensors labeled 831, 832, 833, 834, 835 and 836, respectively. Acoustic Sensors 831, 832, 833, 834, 835 and 836 may be interconnected by a Webbing 839. In the present design, Acoustic Sensors 831, 832, 833, 834, 835 and 836 are oriented at the anterior thoracic ECG positions V1, V2, V3, V4, V5 and V6 respectively, as shown in FIG. 2. The Webbing 839 ensures proper placement of the Acoustic Sensors 831, 832, 833, 834, 835 and 836 across the patients torso, and enables the application of a single pad for multiple readouts.

As previously discussed, Webbing 839 may, in some embodiments, be a cloth mesh or plastic. Alternatively, Webbing 839 may be rigid in nature and include metal or plastics. In some embodiments, Webbing 839 may be connector rods of any suitable material. Webbing 839 functions to maintain the relative positions of the Acoustic Sensors 831, 832, 833, 834, 835 and 836 to one another.

In some embodiments, Acoustic Sensors 831, 832, 833, 834, 835 and 836 may be bell shaped sensor pads, with a microphone or piezo sensor in the vertex of the bell. Additionally, Acoustic Sensors 831, 832, 833, 834, 835 and 836 may include ECG functionality.

Moreover, Acoustic Sensors 831, 832, 833, 834, 835 and 836 may, in some embodiments, additionally provide an active signal through a transducer. In other embodiments, a separate transducer may be utilized to generate the active acoustic signals.

Moreover, perceived signals by the Acoustic Sensors 831, 832, 833, 834, 835 and 836 may enable depth and location triangulation for internal structures when utilizing echo signals.

As previously discussed, in some embodiments, the Chest Pad 830 may be designed in variant sizing for separate body sizes and types. In some embodiments, the Webbing 839 may be elastic as to increase wearer comfort and enable a singular device to be utilized by a wide gamut of individuals.

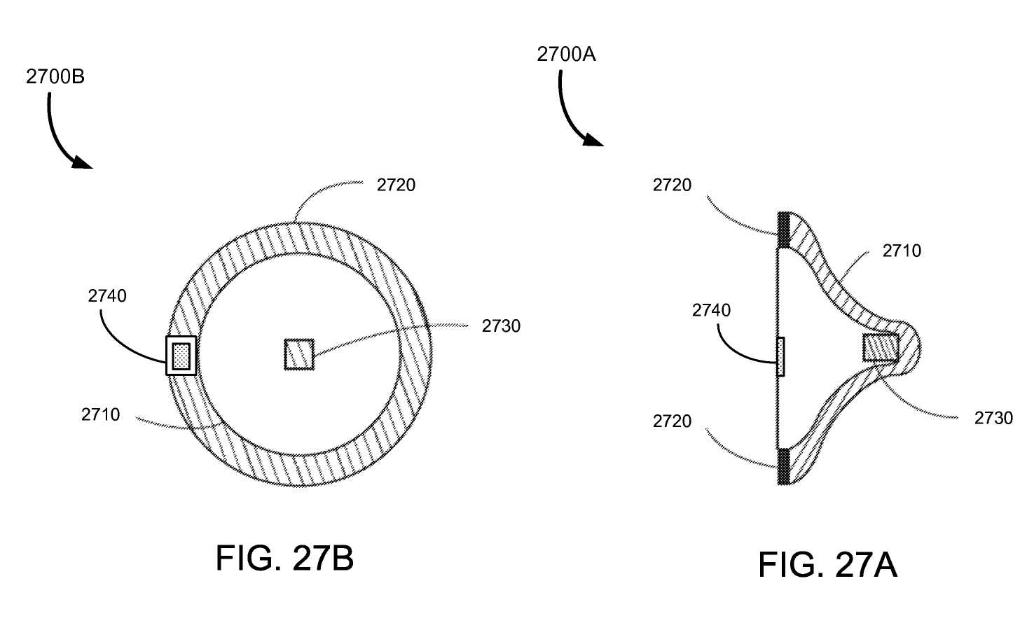

FIG. 27A shows a front view illustrating one embodiment of a chest-patch which combines an ECG Sensor 2720 with a Transducer 2730 and a Pressure Sensor 2740, shown generally at 2700A. Similarly, FIG. 27B shows a side view of the same chest-patch, shown generally at 2700B. In this embodiment, the ECG Sensor 2720 is a conductive ring allowing ECG electrical signal transmission from the base of the Housing 2710. ECG Sensor 2720 may include a sealing membrane to ensure both electrical conduction and mechanical air seal for superior acoustic transmission. Sealing may also be accomplished by an ECG gel in combination with or in place of a sealing membrane. Housing 2710 may be filled with air or fluid to facilitate acoustic transmission.

The Transducer 2730 may, in some embodiments, be a traditional membrane and magnet microphone. Alternatively, Transducer 2730 may be a piezo transducer. Of course additional transducers may be utilized as is known by those skilled in the art.

A piezo Transducer 2730 may be capable of producing an acoustic signal, as well as sensing acoustic waves. Thus, the Transducer 2730, in some embodiments where piezo or similar designs are utilized, may both supply the acoustic signal as well as provide sensory reception. In some alternate embodiments, the Transducer 2730 may be designed to only receive acoustic signals.

A Pressure Sensor 2740 may exist along the seal of the Housing 2710 and the body. The Pressure Sensor 2740 may provide information as to the quality of the seal between the Housing 2710 and the patient's body. Since every application of the chest patch requires a human, there is infallibly some variation in the force of which the chest pad is applied. By recognizing these differences in pad application, via the Pressure Sensor 2740, the acoustic signals received may be additionally calibrated. In some embodiments, an incorrectly applied chest pad may even provide the health care giver with a notification that the pad is defectively applied.

The Pressure Sensor 2740, as illustrated, may include a mechanical transducer or similar mechanism to provide information as to the pressure of the chest pad application. Alternatively, piezo material may be incorporated into the seal region. A tighter application of the chest pad will stretch the seal, thereby deforming the piezo. A voltage proportional to the deformation may be produced, enabling measurement of the force of chest pad application.

It should be noted that in some embodiments the ECG Sensor 2720 may be omitted. Thus the chest pad would include phonographic properties and a pressure calibration. Of course additional sensory components may be included in the chest pad as is desired.

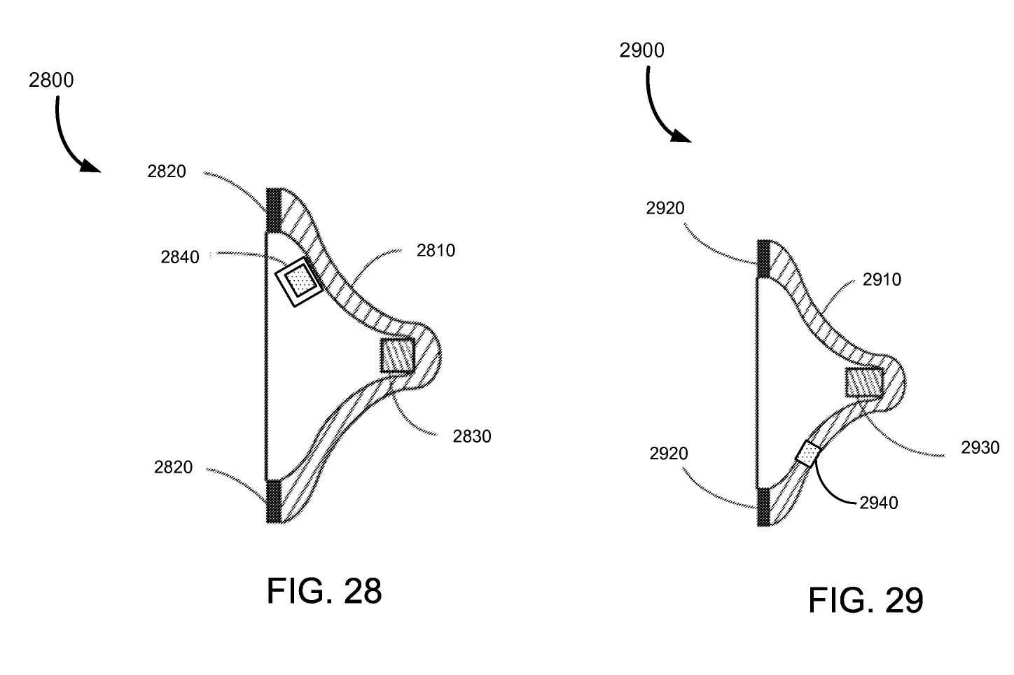

FIG. 28 shows a front view illustrating another embodiment of a chest-patch which combines an ECG sensor with an acoustic transducer and a pressure sensor, shown generally at 2800. Like the previous embodiments this chest pad includes a Housing 2810, ECG Sensor 2820, Transducer 2830 and Pressure Sensor 2840. However, in this embodiment the Pressure Sensor 2840 is located within the interior of the Housing 2810.

In this embodiment, the Housing 2810 may be filled with air or other fluid. The Pressure Sensor 2840 may then measure the fluid pressure within the Housing 2810. As the chest pad is applied to the patient, fluid pressure will increase within the Housing 2810. The degree of fluid pressure increase may be measured by the Pressure Sensor 2840 to provide calibration data. As previously mentioned, the Pressure Sensor 2840 may include a mechanical transducer, or piezo style pressure sensor.

Like previous embodiments, the ECG Sensor 2820 may be omitted. Thus the chest pad would include phonographic properties and a pressure calibration. Of course additional sensory components may be included in the chest pad as is desired.

FIG. 29 shows a front view illustrating yet another embodiment of a chest-patch which combines an ECG sensor with an acoustic transducer and a pressure sensor, shown generally at 2900. Like the previous embodiments, this chest pad includes a Housing 2910, ECG Sensor 2920, Transducer 2930 and Pressure Sensor 2940. However, in this embodiment the Pressure Sensor 2940 is located as integrated within the wall of the Housing 2910.

In this embodiment, the Pressure Sensor 2940 may directly measure deformation of the Housing 2910 associated with the application of the chest pad to the patient. As previously mentioned, the Pressure Sensor 2940 may include a mechanical transducer, or piezo style pressure sensor.

Like previous embodiments, the ECG Sensor 2920 may be omitted. Thus the chest pad would include phonographic properties and a pressure calibration. Of course additional sensory components may be included in the chest pad as is desired.

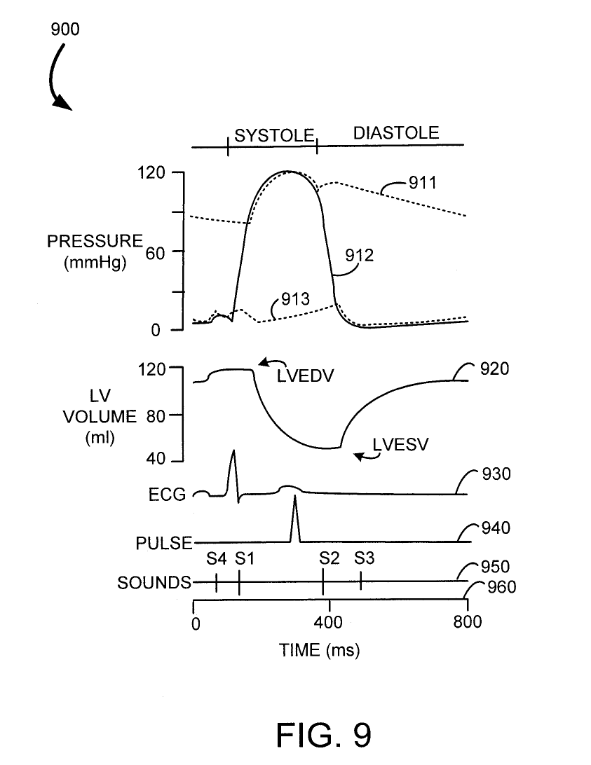

FIG. 9 shows an exemplary diagram of pressure, timing, blood volume and signals associated in a typical cardiac cycle, shown generally at 900.

The cardiac cycle diagram shown depicts changes in aortic pressure (AP) 911, left ventricular pressure (LVP) 912, left arterial pressure (LAP) 913, left ventricular volume (LV Vol) 920, acoustic echo Pulse 940 and heart sounds 950 during a single cycle of cardiac contraction and relaxation. These changes are related in time to the electrocardiogram.

Typically aortic pressure is measured by inserting a pressure catheter into the aorta from a peripheral artery, and the left ventricular pressure is obtained by placing a pressure catheter inside the left ventricle and measuring changes in intraventricular pressure as the heart beats. Left arterial pressure is not usually measured directly, except in investigational procedures. Ventricular volume changes can be assessed in real time using echocardiography or radionuclide imaging, or by using a special volume conductance catheter placed within the ventricle.

A single cycle of cardiac activity can be divided into two basic stages. The first stage is diastole, which represents ventricular filling and a brief period just prior to filling at which time the ventricles are relaxing. The second stage is systole, which represents the time of contraction and ejection of blood from the ventricles.

The Pulse 940 shown is intended to be exemplary in nature. The Pulse 940 may be 10 to 100 microseconds in length. In some embodiments, longer pulses may be utilized. The diagram illustrates a longer Pulse 940 for viewing ease. In yet other embodiments, continuous acoustic signals may be supplied by the acoustic transducer. Additionally, the Pulse 940 may be varied in time across the cardiac cycle as to interleave the Pulse 940 and heart sounds.

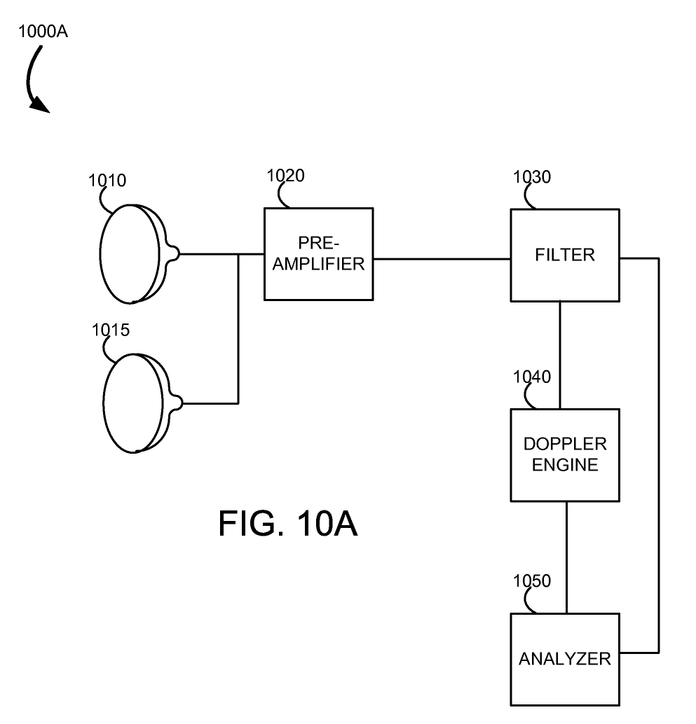

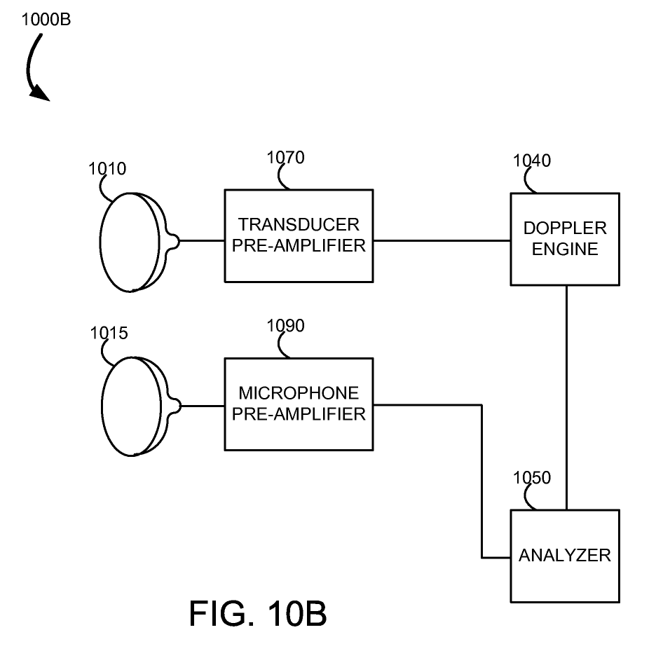

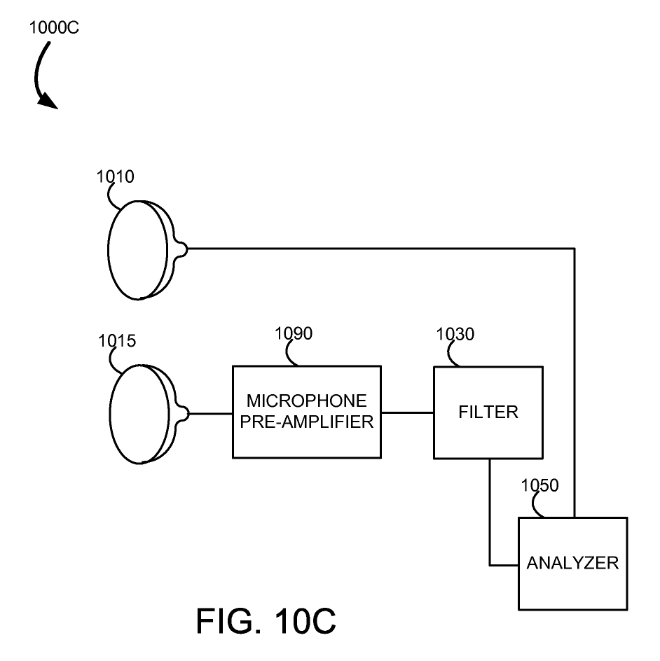

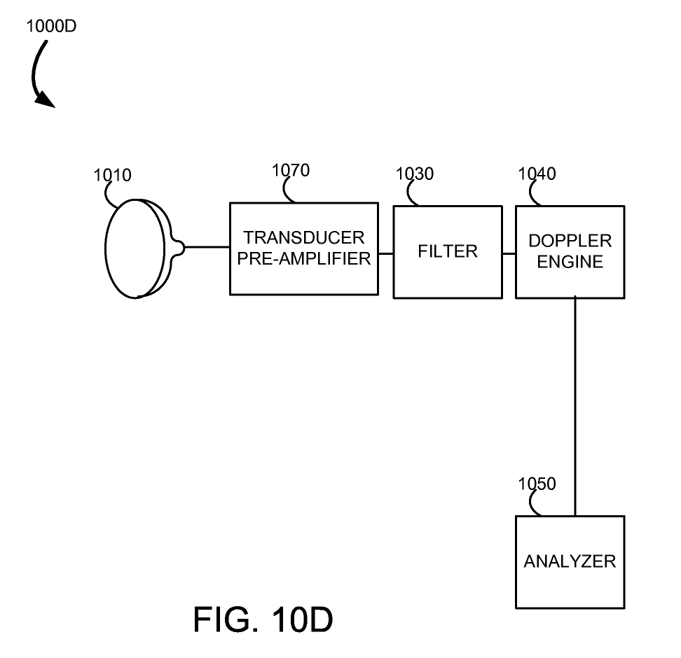

FIGS. 10A through 10D provide exemplary functional diagrams of the auscultatory device. Additional embodiments are possible, and it is intended that the spirit of these additional embodiments is included in the exemplary embodiments.

FIG. 10A shows a functional block diagram of one embodiment of the auscultatory device shown generally at 1000A. The Acoustic Transducer 1010 may be any of the sensory devices illustrated in FIGS. 3A to 7, as well as any sensory design as is known by those skilled in the art. The Acoustic Transducer 1010 may couple to a Pre-amplifier 1020. An Acoustic Sensor 1015 may be any acoustic sensor designed to be responsive to heart sounds, such as a Stethoscope 120. The Acoustic Sensor 1015 may likewise couple to the Pre-amplifier 1020. In some embodiments, the Acoustic Sensor 1015 and Acoustic Transducer 1010 may be housed within the same unit. Additionally, in some embodiments, a single sensor may incorporate both the Acoustic Sensor 1015 and Acoustic Transducer 1010.

The Pre-amplifier 1020 may amplify the source signal to line signal levels. Additional equalizing and tone control may be performed by the Pre-amplifier 1020 as well. In some embodiments, where the echo signal received from the Acoustic Transducer 1010 far outweighs the heart signals from the Acoustic Sensor 1015, additional protective circuitry may be utilized in order to preserve the heart sound signals.

The Pre-amplifier 1020 couples to a Filter 1030, which is enabled to separate the signals relating to heart sounds from those received from the echo of the generated acoustic signals. As previously discussed, Heart sounds are typically low in frequency, e.g., typically 10 to 500 Hz. The generated acoustic signals may be in the MHz range. As such, high pass and low pass filters may easily distinguish between sounds originating from the heart, and those echoing from the generated signals from the acoustic transducer.

The Filter 1030 may be coupled to a Doppler Engine 1040 and an Analyzer 1050. The Doppler Engine 1040 may, in some embodiments, receive the echo signals separated by the Filter 1030, while the heart sounds are sent directly to the Analyzer 1050. The Doppler Engine 1040 may be enabled to deduce the speed at which the valve leaflet is moving with respect to the sound wave by detecting Doppler shifting. Alternatively, another way to deduce the speed is to measure the distance traversed by the moving leaflet, and knowing the time interval between the two measurements and computing leaflet speed. In such embodiments, the Doppler Engine 1040 may be unnecessary. The former involves more sophisticated electronics; the latter is simpler in implementation but may be less precise. Additional methods of determining valve leaflet speed may be utilized as is known by those skilled in the art.

The Doppler Engine 1040 also allows for blood flow to be detected and further helps to characterize any heart sound component caused by regurgitant jet. Additionally, Doppler processing increases the accuracy and robustness of determining the spatial (which valve) and temporal (systole or diastole) origin of a murmur.

The Analyzer 1050 may provide display and analysis of the received signals. Such analysis includes, but is not limited to S1/S2 sound ratios and heart sound calibration utilizing the ratio of S1 and the attenuated sound (Sc), the intensity ratio (S1/Sc).

FIG. 10B shows a functional block diagram of another embodiment of the auscultatory device shown generally at 1000B. The Acoustic Transducer 1010 may be any of the sensory devices illustrated in FIGS. 3A to 7, as well as any sensory design as is known by those skilled in the art. The Acoustic Transducer 1010 may couple to a Transducer Pre-amplifier 1070. An Acoustic Sensor 1015 may be any acoustic sensor designed to be responsive to heart sounds, such as a Stethoscope 120. The Acoustic Sensor 1015 may be couple to the Microphone Pre-amplifier 1090. In some embodiments, the Acoustic Sensor 1015 and Acoustic Transducer 1010 may be housed within the same unit.

The Transducer Pre-amplifier 1070 may amplify the perceived pulse echo signal to a line signal levels. Additional equalizing and tone control may be performed by the Transducer Pre-amplifier 1070 as well. Likewise, the Microphone Pre-amplifier 1090 may amplify the perceived heart sound signal to a line signal levels. Additional equalizing and tone control may be performed by the Microphone Pre-amplifier 1090 as well. The utilization of two channels dedicated to heart sounds and pulse echo signals separately eliminates the requirement for filters.

The Transducer Pre-amplifier 1070 may be coupled to a Doppler Engine 1040. As previously stated, the Doppler Engine 1040 may be enabled to deduce the speed at which the valve leaflet is moving with respect to the sound wave by detecting Doppler shifting. Alternatively, as previously discussed, alternative methods for determining valve leaflet speed may be utilized.

The Doppler Engine 1040 also allows for blood flow to be detected and further helps to characterize any heart sound component caused by regurgitant jet. Additionally, Doppler processing increases the accuracy and robustness of determining the spatial (which valve) and temporal (systole or diastole) origin of a murmur.

The Microphone Pre-amplifier 1090 and the Doppler Engine 1040 couple to the Analyzer 1050 which may provide display and analysis of the received signals. Such analysis includes, but is not limited to S1/S2 sound ratios and heart sound calibration utilizing the ratio of S1 and the attenuated sound (Sc), the intensity ratio (S1/Sc).

FIG. 10C shows a functional block diagram of yet another embodiment of the auscultatory device shown generally at 1000C. The Acoustic Transducer 1010 may be any acoustic generation device, such as speaker or piezo, as is known by those skilled in the art. An Acoustic Sensor 1015 may be any acoustic sensor designed to be responsive to heart sounds and the acoustic signal generated by the Transducer 1010, such as a Stethoscope 120. The Acoustic Sensor 1015 may be couple to the Microphone Pre-amplifier 1090. In some embodiments, the Acoustic Sensor 1015 and Acoustic Transducer 1010 may be housed within the same unit.

The Microphone Pre-amplifier 1090 may amplify the perceived heart sound signal and transduced signal to a line signal levels. Additional equalizing and tone control may be performed by the Microphone Pre-amplifier 1090 as well. A Filter 1030 may separate the perceived heart sound signal from the transduced signal. Alternatively, in some embodiments, time interleaving may be utilized in order to temporally separate heart signals from transduced signals.

The Transducer 1010 and the Filter 1030 couples to the Analyzer 1050 which may provide display and analysis of the received signals. Such analysis includes, but is not limited to S1/S2 sound ratios and heart sound calibration utilizing the ratio of S1 and the attenuated sound (Sc), the intensity ratio (S1/Sc).

FIG. 10D shows a functional block diagram of yet another embodiment of the auscultatory device shown generally at 1000D. The Acoustic Transducer 1010 may be any of the sensory devices illustrated in FIGS. 3A to 7, as well as any sensory design as is known by those skilled in the art. In this and similar embodiments, the Transducer 1010 may both generate a pulse echo as well as provides sensory ability. The Acoustic Transducer 1010 may couple to a Transducer Pre-amplifier 1070. Transducer 1010 may be designed to be responsive to heart sounds as well as the generated pulse echo.

The Transducer Pre-amplifier 1070 may amplify the perceived pulse echo signal and heart sound signal to a line signal levels. Additional equalizing and tone control may be performed by the Transducer Pre-amplifier 1070 as well. A Filter 1030 may separate the perceived heart sound signal from the transduced signal. Alternatively, in some embodiments, time interleaving may be utilized in order to temporally separate heart signals from transduced signals.

The Filter 1030 may be coupled to a Doppler Engine 1040. As previously stated, the Doppler Engine 1040 may be enabled to deduce the speed at which the valve leaflet is moving with respect to the sound wave by detecting Doppler shifting. Alternatively, as previously discussed, alternative methods for determining valve leaflet speed may be utilized.

The Doppler Engine 1040 also allows for blood flow to be detected and further helps to characterize any heart sound component caused by regurgitant jet. Additionally, Doppler processing increases the accuracy and robustness of determining the spatial (which valve) and temporal (systole or diastole) origin of a murmur.

The Doppler Engine 1040 couples to the Analyzer 1050 which may provide display and analysis of the received signals. Such analysis includes, but is not limited to S1/S2 sound ratios and heart sound calibration utilizing the ratio of S1 and the attenuated sound (Sc), the intensity ratio (S1/Sc), speed and motion analysis and localization of sound sources.

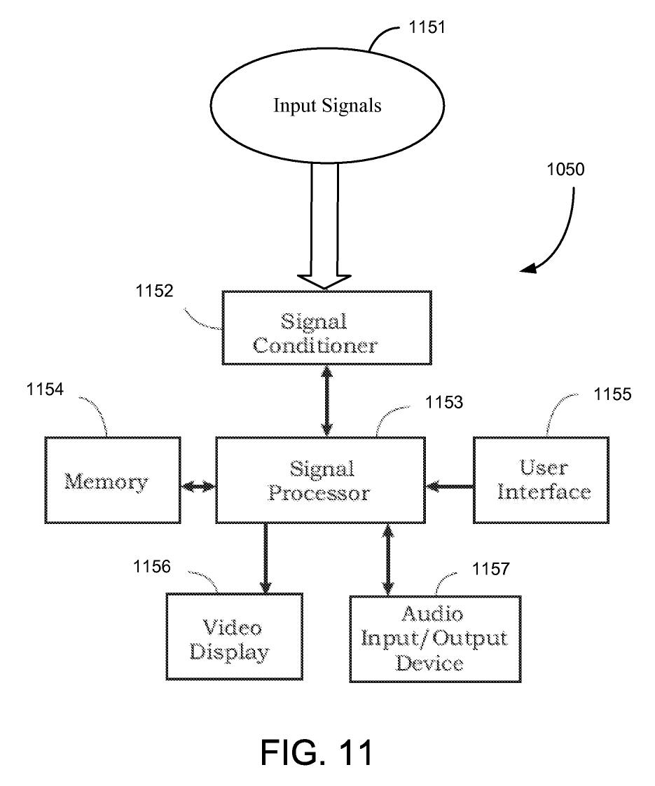

FIG. 11 shows an illustration of a functional block diagram for the Analyzer 1050 in accordance with an embodiment of the present invention. Analyzer 1050 includes a Signal Conditioner 1152, Signal Processor 1153, Memory 1154, User Interface 1155, Video Display 1156 and Acoustic Input/Output Device 1157.

Input Signals 1151 are received from the Doppler Engine 1040, Filter 1030 and Microphone Pre-Amplifier 1090. Such raw Input Signals 1151 are processed through a Signal Conditioner 1152. Conditioned signals may then be analyzed by the Signal Processor 1153. Signal Processor 1153 may couple to Memory 1154, User Interface 1155, Video Display 1156 and Acoustic Input/Output Device 1157.

Memory 1154 can be fixed or removal memory, and combinations thereof. Examples of suitable technologies for memory 1154 include solid-state memory such as flash memory, or a hard disk drive.

User interface 1155 can be a keypad, a keyboard, a thumbwheel, a joystick, and combinations thereof. Video display 1156 can be an LCD screen, or can be an LED display or a miniature plasma screen. It is also possible to combine video display 1156 with user interface 1155 by use of technologies such as a touch screen. Contrast and brightness control capability can also be added to display 1156.

Acoustic input/output (I/O) device 1157 includes a microphone, and speakers, earphones or headphones, any of which can be internal or external with respect to Analyzer 1050. It is also possible to use wireless acoustic I/O devices such as a Bluetooth-based headset. Volume control may also be provided.

Logical couplings of these components may be otherwise organized as is advantageous. Additionally, alternate or additional components may be included in the Analyzer 1050.

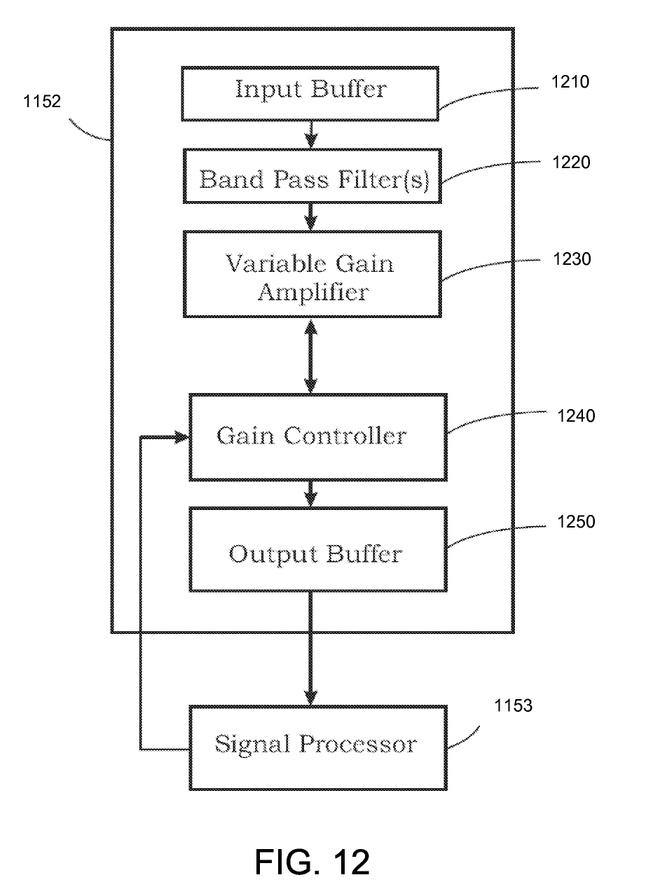

FIG. 12 provides a detailed block diagram illustrating heart sound Signal Conditioner 1152 which includes an Input Buffer 1210, one or more Band Pass Filter(s) 1220, a Variable Gain Amplifier 1230, a Gain Controller 1240 and an Output Buffer 1250. Output buffer 1250 is coupled to Signal Processor 1153 which in turn is coupled to Gain Controller 1240.

In some embodiments, Filter 1220 is a 4ₜₕ order Butterworth pass band of 5 Hz to 2 kHz which limits the analysis of the heart sound signal to frequencies less than 2 kHz, thereby ensuring that all frequencies of the heart sounds are faithfully captured and at the same time eliminating noise sources that typically exist beyond the pass band of Filter 1220. Of course additional Filters 1220 may be utilized as is desired.

Variable Gain Amplifier 1230 of Signal Conditioner 1152 serves to vary the signal gain based on a user-selectable input parameter, and also serves to ensure enhanced signal quality and improved signal to noise ratio. The conditioned heart sound signal after filtering and amplification is then provided to Signal Processor 1153 via Output Buffer 1250.

Additional signal conditioning components may be incorporated into the Signal Conditioner 1152 as is desired. For example, in some embodiments, a component for eliminating low amplitude noise signals may be utilized.

### Self Calibration

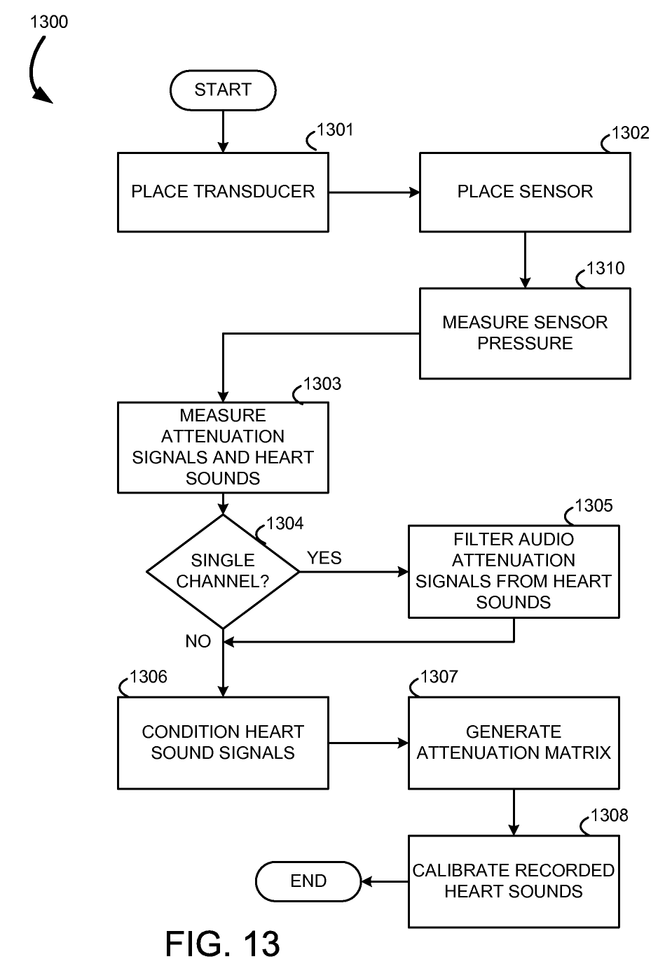

FIGS. 13 to 15 provide methods and processes for the calibration of heart sound measurements by use of an active transduction signal. Such a signal may be measured to produce attenuation values and subsequent heart sound calibrations. Moreover, pressure sensors on chest pad, as illustrated at FIGS. 27 to 29, may be further utilized in order to calibrate the received heart sounds dependent upon chest pad placement. Heart sound calibration has diagnostic use, and provides an ability to perform cross-patient heart sound analyses.

FIG. 13 shows an exemplary process for self calibration of heart signals utilizing an embodiment of the auscultatory device, shown generally at 1300. Such a process may be performed automatically by the auscultatory device, without need of user input. Such a process may equalize heart sounds from a range of patients. Additionally, calibrated heart signals may be utilized in a range of subsequent diagnostic processes, such as Ejection Fraction determination.

In some embodiments, there are two ways to calibrate S1, each with its own advantages and disadvantages. The first includes calibrating S1 with S2. The advantage of this method is that each patient will calibrate him/herself, since the body equally attenuates both sounds and there is no additional need to work out different attenuations for different people. A simple comparison of a patient's S1 intensity to their S2 intensity may be utilized to produce meaningful diagnostic ratios. The disadvantage of this method is that S2 itself may be affected by a heart condition and may be unsuitable.

Secondly, calibration of the S1 may be performed by utilizing the attenuation values recorded. In some embodiments, multiple tones may be utilized, at various frequencies in the first heart sound spectrum. The advantage of this method is that the attenuation of the tones should be representative on each subject of sound attenuation in their body. There is no bias regarding their cardiac health, as is the case with calibration by S2. In some embodiments, the transmission tones are just simple tones; however more complex attenuation signals may be utilized.

The process begins at step 1301 where the transducer is placed upon the patient at the Transducing Location 141. Any transducer disclosed in FIGS. 3 to 8c, 27 to 27 may be utilized. In some embodiments, such transducers produce a constant active signal during calibration. A sensor may be placed at the Sensing Location 131 at step 1302.

The process then proceeds to step 1310 where pressure of chest pad application to the patient is measured. This pressure measurement may be made by the pressure sensor as illustrated in any of the exemplary illustrations of FIGS. 27A to 29. Chest pad application is inherently variable, and a pressure sensor may be utilized to aid in signal calibration by accounting for this variability in pad application. It should be noted, however, that this calibration is optional. Thus, in some embodiments, step 1310 may be omitted in systems that are not enabled to measure chest pad application pressure.

The sensor may receive signals that pass through the patient's body. These received signals are measured at step 1303. As addressed earlier, the transduced signals may be within physiological frequency ranges. Additional frequencies, steeped frequencies and variable frequencies may also be utilized. A single sensor may be utilized to measure both generated attenuation signal as well as patient heart sounds. Alternatively, additional sensors may be utilized to measure heart sounds and attenuation signals. Sensor(s) responsive range is calibrated to be sensitive to attenuation signal range and physiological sound ranges.

At step 1304, a determination is made as to whether heart sounds and attenuation signals are on the same channel. Such is the case when attenuation signal and heart sounds are perceived by a common sensor. If these signals share a single channel, the signals may be filtered at step 1305. Filtering may be performed by band pass filtering, in the instances where attenuation signal is of a separate frequency range than heart sounds. Alternatively, filtering may include a very narrow band pass filter for the attenuation frequency when the attenuation signal is within a physiological range. The signal is then conditioned at step 1306.

If, at step 1304, the attenuation signal and the heart sounds are on separate channels, then the signal is conditioned at step 1306. Separate channels for the heart signals and attenuation signals is achieved when separate frequency ranges are utilized for the attenuation signal as compared to the heart sound frequency, and separate sensors are utilized for the measuring of the respective signals. The sensors may, in some embodiments, be responsive to the particular frequency range they are measuring thereby providing an intrinsic filtering.

After signal conditioning, the process proceeds to step 1307, where an attenuation matrix is generated. To generate the matrix, the signal amplitude for each transducer/sensor location is compiled.

Then at step 1308, the measured heart sounds may be calibrated by using the attenuation matrix, along with the pressure data from the chest pad pressure sensors. The S1 may be calibrated by the use of any combination of the values in the attenuation matrix along with the pressure data.

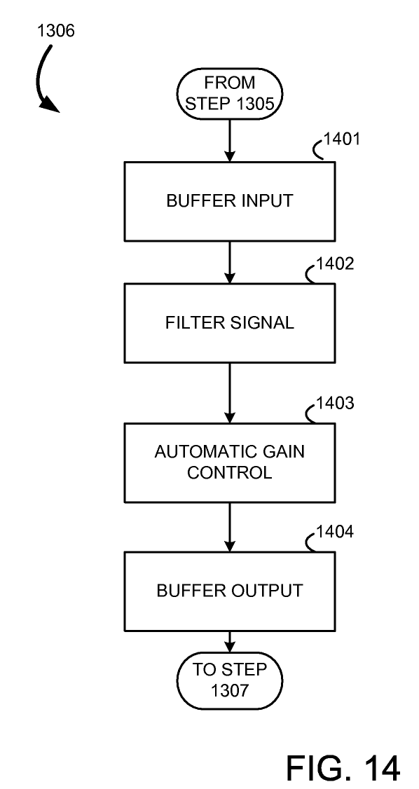

FIG. 14 shows an exemplary process for signal conditioning of heart signals utilizing an embodiment of the auscultatory device, shown generally at 1306. Signal conditioning may occur at the Signal Conditioner 1152.

The process begins from step 1305 from FIG. 13. The process then proceeds to step 1401 where the input signal is buffered. Buffering occurs at the Input Buffer 1201. Then, at step 1402, the signal may undergo additional filtering. The filtering operations may involve simple filters, for example a straightforward analog Butterworth nth order bandpass/lowpass/highpass filter. It is conceivable that wavelet operations, which by their nature divide up the signal into various frequency bands, can also be used to carry out measurements on the heart sound signal. Additional filtering techniques may be employed as is known by those skilled in the art. Filtering may occur at the Band Pass Filter(s) 1202.

The process then proceeds to step 1403 where gain may be automatically controlled. A Variable Gain Amplifier 1203 in conjunction with the Gain Controller 1204 may effectuate automatic gain control.

The process then proceeds to step 1404 where the output is buffered. The Output Buffer 1205 may perform this operation. Additional signal conditioning steps may be performed as is known by those skilled in the art. The process then ends by proceeding to step 1307.

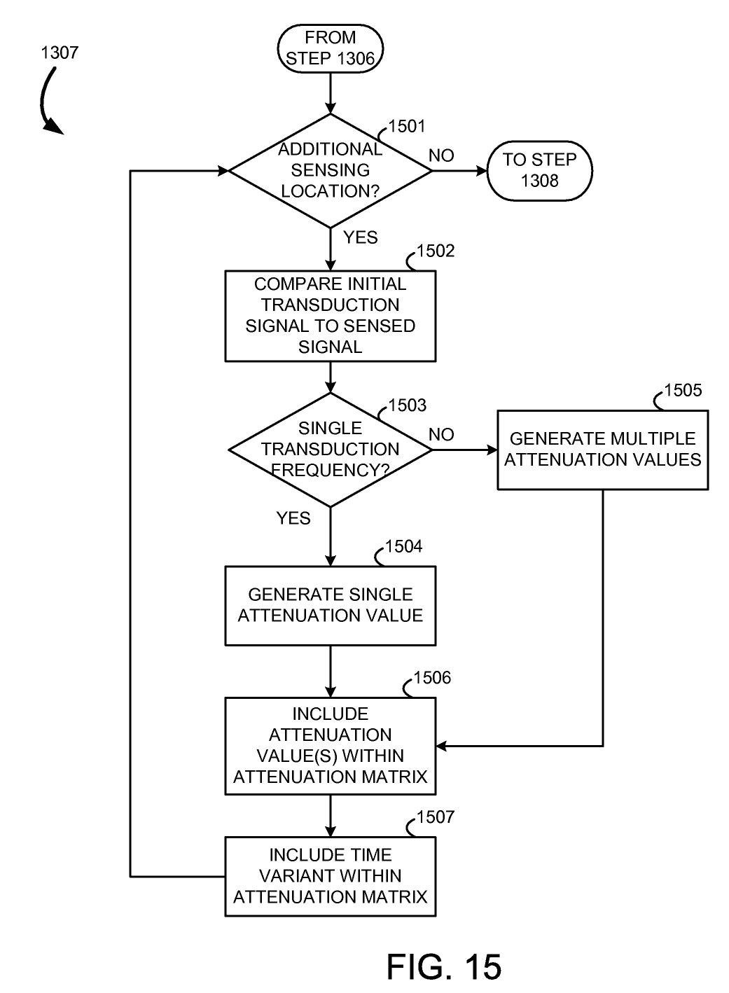

FIG. 15 shows an exemplary process for generating the attenuation matrix utilizing an embodiment of the auscultatory device, shown generally at 1307. The use of an attenuation matrix is but one suitable method of representing attenuation signal data for use with calibration. As such, the present method is intended to be exemplary in nature. No limitations upon the present invention are suggested by the disclosure of attenuation matrix generation. Moreover, additional representations, such as a single attenuation value, an attenuation value list or three dimensional attenuation value matrices may be utilized.

The process begins from step 1306. Then at step 1501 an inquiry is made whether an additional sensing location is desired. If at step 1306 an additional sensing location is desired, then the process proceeds to step 1502, where the known initial transduction signal is compared to the perceived attenuation signal. The initial transduction signal may, in some embodiments, include a constant sinusoidal sound signal. Alternative sound waveforms, frequencies and durations may be utilized as is desired. The difference between the known initial transduction signal and the perceived attenuation signal provides information about internal structures along the sound wave path.

Then at step 1503, an inquiry is made as to whether the transduction signal was a single frequency signal. If so, then at step 1504 a single attenuation value may be generated. The single attenuation value may then be added to an attenuation matrix in step 1506.

Else, if at step 1503, the initial transduction signal was not of a single frequency, then the process proceeds to step 1505 where multiple attenuation values are generated. The multiple attenuation values may then be added to an attenuation matrix in step 1506.

Then in step 1507, a time variant value may be added to the matrix. The time variant value is the time differential between signal transduction and perceived attenuation signal measurement.

The process then proceeds back to step 1501, where an inquiry is made whether an additional sensing location is desired. In this way the process will be repeated for each sensing location desired. Attenuation values for each sensing location may be compiled into the attenuation matrix. Once all sensing locations have been measured the process ends.

In this way heart sounds may be calibrated for by utilizing an active transduction signal that passes through the patient's chest cavity. Additional methods for heart sound calibration may additionally be utilized, including both invasive and non-invasive procedures.

### Pulsed Echo

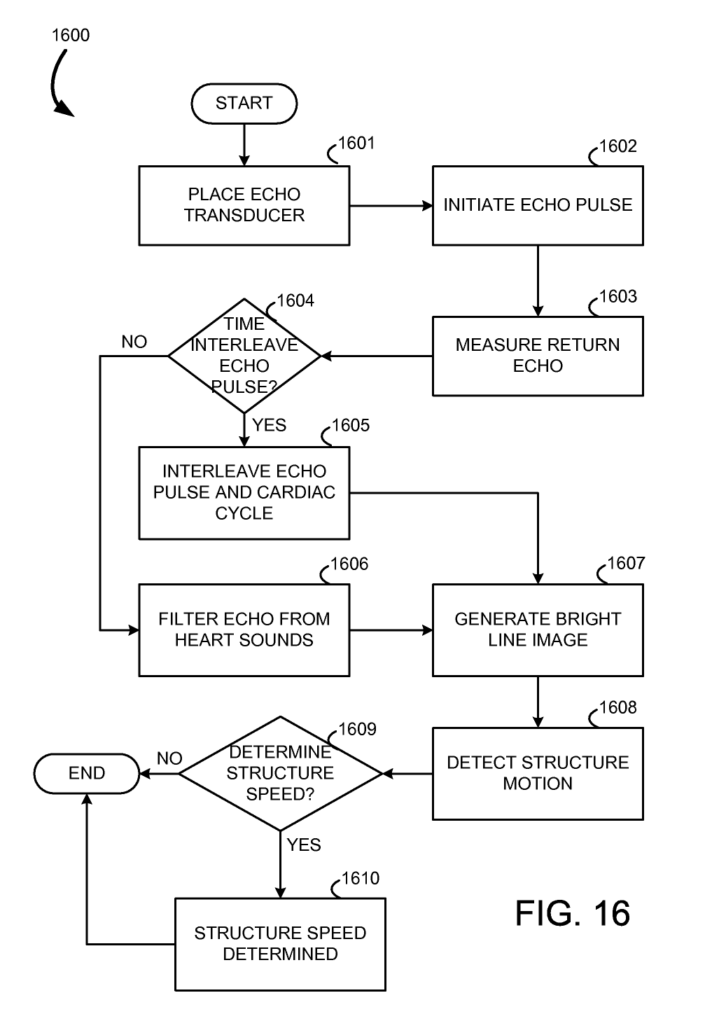

FIGS. 16 to 18 further illustrate methods for pulsed echo cartographic analysis. Pulsed echo refers to the usage of pulsed acoustics to provide a reflective “image” of internal structures. In some embodiments, the echo pulse may be of higher frequencies as to provide adequate resolutions. The ability to sense structure motion, location and speed of motion makes the pulsed echo of particular use in identifying pathologies such as a faulty valve.

FIG. 16 shows an exemplary process for pulsed echo utilizing an embodiment of the auscultatory device, shown generally at 1600. The process begins at step 1601 where the pulsed echo transducer is placed in the transducer position on the patient's torso. Then, at step 1602, an echo pulse is induced. The echo pulse, in some embodiments, may be a few microseconds up to few tens of microseconds in duration. Operating in MHz range provides adequate resolution. Echo pulses may be repeated as necessary.

At step 1603, the return echo is measured. Then, at step 1604, an inquiry is made whether to utilize time interleaving. If time interleaving is desired, then the process proceeds to step 1605 where echo pulses and cardiac signals are interleaved as to minimize the potential loss of signal data. Time interleaving separates heart signals from echo pulse temporally, thereby removing the need for additional filtering. Time interleaving may additionally be useful when the echo pulse saturates the received signals. Then at step 1607, a bright line image is generated. The bright line image is a representation of the structures encountered by the pulse echo.

Else, if at step 1604 time interleaving is not desired, the process then proceeds to step 1606, where the heart signals are filtered from the echo signals. Since, in some embodiments, the echo pulse is of much higher frequency than heart sounds, a simple high pass filtering may be utilized to separate heart signals from the echo pulse. Then at step 1607, a bright line image is generated. The bright line image is a representation of the structures encountered by the pulse echo.

Then at step 1608, structure motion is identified. An inquiry is made if moving structure speed is to be determined at step 1609. In some embodiments, speed of moving structures may be automatically generated. In other embodiments, speed determination may be performed on a case-by-case basis. In such embodiments, the user physician may select a mode for speed capture on the auscultatory device. If speed of the moving structure is desired, the process proceeds to step 1610 where the structure speed is identified. A typical structure which speed may be measured includes heart valve leaflet closure rates, blood flow, heart wall constriction or any additional moving structure. After structure speed is determined, the process ends. Else, if at step 1609 structure speed is not a required measurement, the process ends.

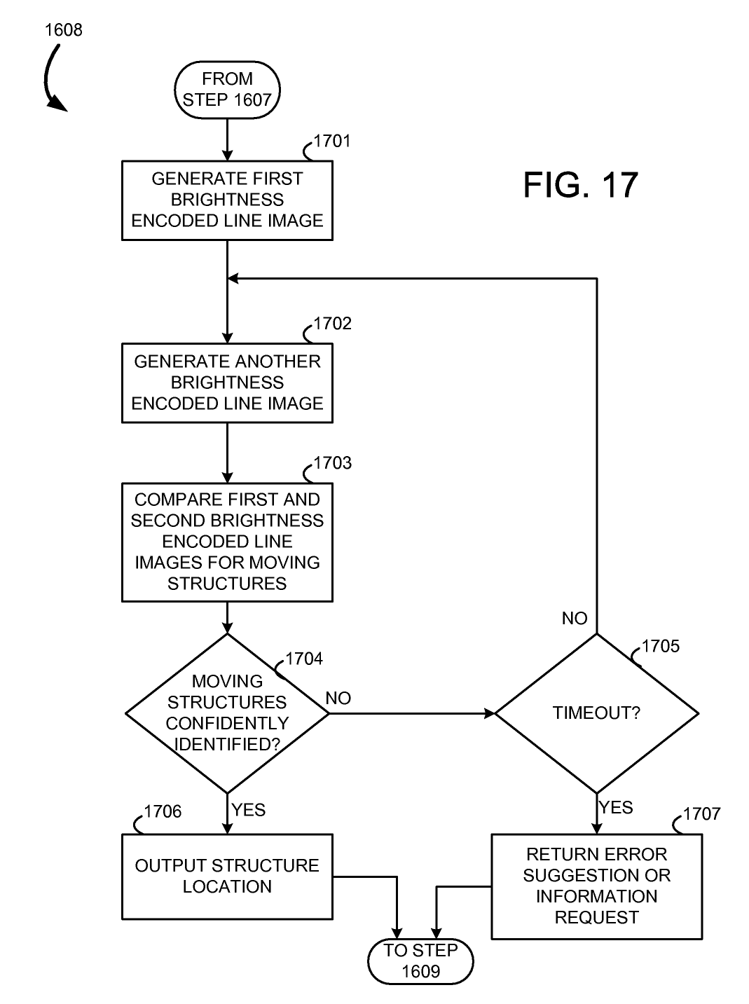

FIG. 17 shows an exemplary process for motion detection in pulsed echo utilizing an embodiment of the auscultatory device, shown generally at 1608. A brightness line image generated from the received acoustic signals provides a representation for the structures along the acoustic path. By maintaining a fixed acoustic path, and repeatedly sensing the structures, motion may be identified and tracked. A heart valve is in motion with respect to the patient's chest wall, thus the distance of the valve to the chest wall may be deduced. Such a deduction may accurately be used to enable the calibration of the heart sound of that particular patient to his chest size or attenuation characteristics (the amount of subcutaneous fat, for example).

Motion analysis helps to orient the heart sound to the particular valve as indicated by the motion trace and can achieve better isolation of particular disease signature of the heart sound associated with that particular valve.

The process begins from step 1607. At step 1701, a first brightness encoded image is generated. This first image is generated with the sensor fixed to the patient's chest. Thus, the image provided is stationary in relation to patient's chest wall. Then at step 1702, another brightness encoded image is generated. Likewise, this additional image is generated with the sensor fixed to the patient's chest. Thus, the image provided is stationary in relation to patient's chest wall. The two images are compared for moving structures at step 1703. Since both images “look” at the same space related to the patient's chest wall, discrepancies between the two brightness encoded images is a result of movement of the structure. Additionally, pulse echo timing and orientation may additionally provide structure location information. Thus, the moving structures location may be likewise identified.

At step 1704, an inquiry is made whether the moving structure is adequately identified. A statistical analysis of confidence levels, as measured by a threshold, may be utilized to determine this. For example, if the auscultatory device is calibrated such that a greater than 75% identification of moving structures is required, and the brightness encoded images identify a moving structure 50% of the time, the auscultatory device may determine that the structure is not adequately identified. In such a circumstance, the process then proceeds to step 1705 where an inquiry is made whether moving structure identification has timed out. If the process has not timed out, then the process may return to step 1702 where an additional brightness encoded image is generated in an attempt to clarify the identification. The process then continues the cycle of comparison, confidence inquiry, etc.

Else, if at step 1705 the process for determining the moving structure has timed out, then the process proceeds to step 1707, where an error message is generated. Such an error message may provide either an information request or suggestion. For example, if the sensor is not pointing in a stable fashion due to hand motion, etc., it may indicate repositioning or provide feedback to the user and likewise indicate when the sensor is pointing accurately at the moving structure. The process then ends by proceeding to step 1609.

Otherwise, if at step 1704 the moving structure is adequately identified, then the process may output the moving structure's location at step 1706. The process then ends by proceeding to step 1609.

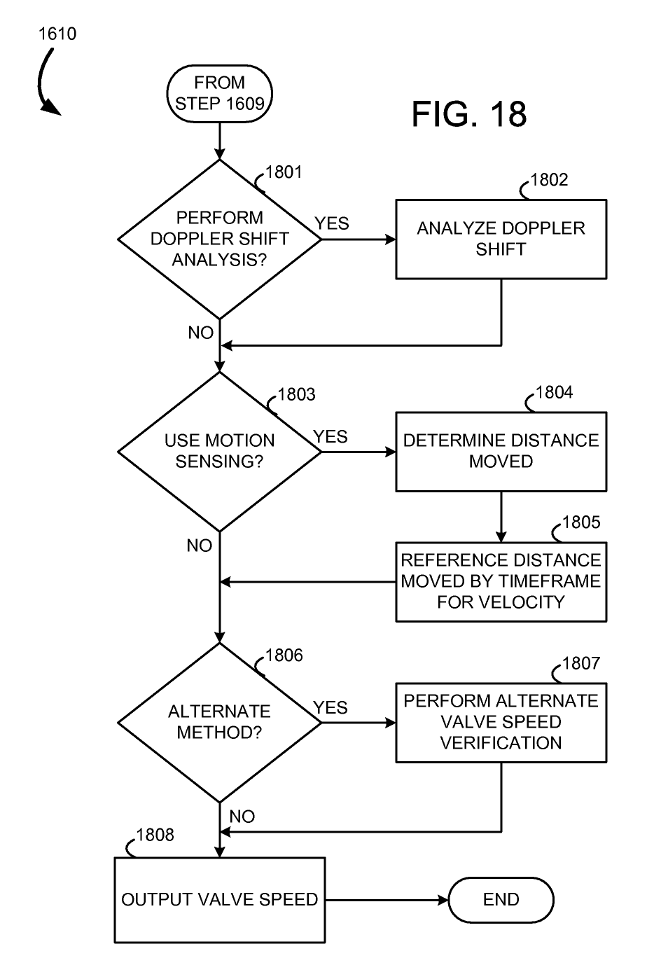

FIG. 18 shows an exemplary process for structure speed detection in pulsed echo utilizing an embodiment of the auscultatory device, shown generally at 1610. The illustrated method includes utilizing a motion trace, Doppler shift detection and alternate methods. In some embodiments, there may be limitations on hardware available, such as Doppler processors. In these embodiments the available hardware may dictate speed determination decisions.

The process begins from step 1609. Then at 1801 an inquiry is made whether to perform a Doppler shift analysis. If a Doppler shift analysis is desired, then the process proceeds to step 1802 where the shift analysis is performed. As the pulse reflects from a moving structure, the return echo will have shifted frequency as related to the speed of the moving structure. A Doppler Engine 1040 may measure the amount of frequency shift in order to determine structure speed. The process then progresses to step 1803 where an inquiry is made whether to determine structure speed by motion tracking.

Else, if at step 1801 a Doppler shift analysis is not performed, then the process progresses to step 1803 where an inquiry is made whether to determine structure speed by motion tracking. Motion tracking for speed determination is simpler than Doppler analysis and requires less hardware, however it tends to be less precise. In some embodiments, motion tracking may be performed in conjunction with Doppler analysis for speed confirmation. If motion tracking for speed determination is desired, then the distance the structure has moved is determined at step 1804. The location information generated during motion detection may be utilized to compute distance traveled. Distance may then be referenced by time taken to travel said distance to generate structure velocity, at step 1805. Then the process proceeds to step 1806 where an inquiry is made whether to determine structure speed by alternate methods.

Otherwise, if at step 1803 motion tracking for speed determination is not desired then the process proceeds to step 1806 where an inquiry is made whether to determine structure speed by alternate methods. Alternate methods may include invasive optical readings, radioactive tagging or any alternate method as is known by those skilled in the art for speed detection. If the alternate method is desired, then it may be performed at step 1807. The speed value is then output at step 1808.

Else, if at step 1806 determining structure speed by alternate methods is not desired, then the process continues directly to step 1808 where speed values are output. Speed value output may include average speed values, maximum and minimum structure speed, and any additional statistical information on structure speed as is desired. The process then ends.

Pulsed echo techniques have particular implications for diagnosis of conditions such as heart murmurs and characterization of any heart sound component caused by regurgitant jet. In heart murmurs, sound location in relation to specific heart valves, valve leaflet closure speed, and blood flow speeds are of particular importance for proper characterization and diagnosis of the ailment. Pulsed echo's ability to locate moving structures, such as heart valves, and determine structure speed is ideal for aiding these heart murmur diagnosis.

Additionally, pulsed echo methods may provide tissue characterization by determination of the distance of the valve to the chest wall. Said distance information may be utilized to calibrate the heart sound of that particular patient to his chest size or attenuation characteristics (the amount of subcutaneous fat, for example). Thus pulsed echo, in conjunction with attenuation information may be utilized to further provide detailed and accurate calibrations of perceived heart sounds.

### Cardiac Contractility Analysis through Calibrated Heart Sounds

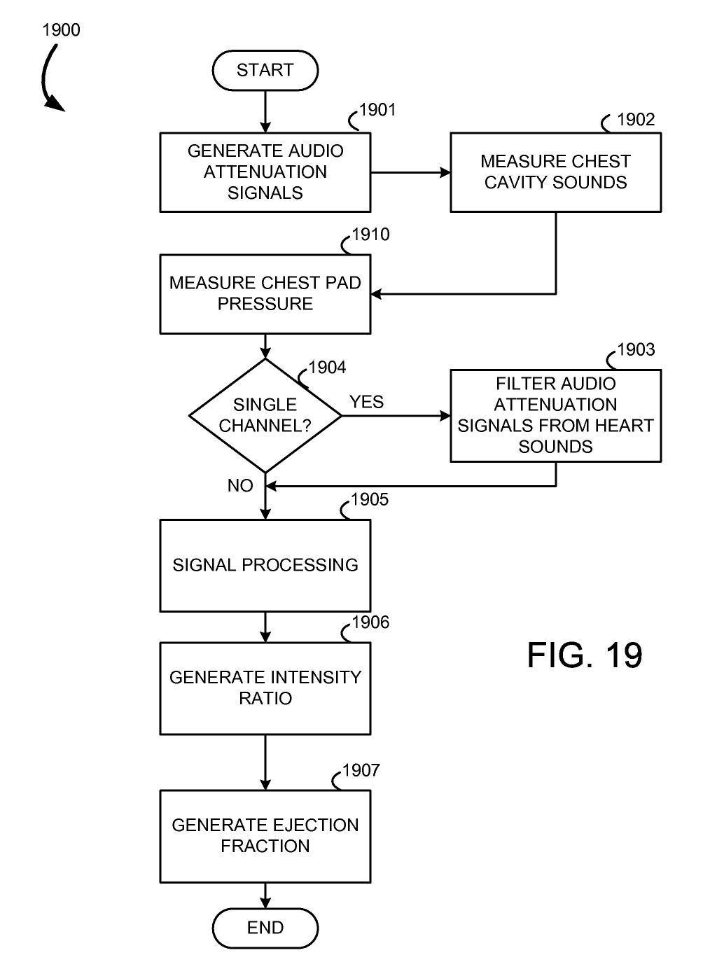

FIGS. 19 to 26 provide exemplary methodologies and examples of the utilization of the auscultatory device to determine cardiac contractility for heart patients. Cardiac contractility may include the rate of change in pressure in a heart (dP/dt), as well as ejection fraction (EF) of the heart.

Ejection Fraction (EF) is the fraction of blood pumped out of a ventricle with each heart beat. The term ejection fraction applies to both the right and left ventricles; one can speak equally of the left ventricular ejection fraction (LVEF) and the right ventricular ejection fraction (RVEF). Without a qualifier, the term ejection fraction refers specifically to that of the left ventricle.

By definition, the volume of blood within a ventricle immediately before a contraction is known as the end-diastolic volume. Similarly, the volume of blood left in a ventricle at the end of contraction is end-systolic volume. The difference between end-diastolic and end-systolic volumes is the stroke volume, the volume of blood ejected with each beat. Ejection fraction (EF) is the fraction of the end-diastolic volume that is ejected with each beat; that is, it is stroke volume (SV) divided by end-diastolic volume (EDV). In a healthy 70-kg (154-lb) man, the SV is approximately 70 ml and the left ventricular EDV is 120 ml, giving an ejection fraction of 70/120, or 58%. Right ventricular volumes being roughly equal to those of the left ventricle, the ejection fraction of the right ventricle is normally equal to that of the left ventricle within narrow limits.

Damage to the muscle of the heart (myocardium), such as that sustained during myocardial infarction or in cardiomyopathy, impairs the heart's ability to eject blood and therefore reduces ejection fraction. Likewise, such damage will result in a lower chance in pressure within the left ventricle throughout the cardiac cycle. This reduction in the ejection fraction and rate of change in pressure in a heart can manifest itself clinically as heart failure. The ejection fraction and rate of change in pressure in a heart are some of the most important predictors of prognosis; those with significantly reduced ejection fractions and reduced rate of change in pressure in a heart typically have poorer prognoses.

FIG. 19 shows an exemplary process for using the auscultatory device to determine cardiac contractility, shown generally at step 1900. Such a process may be utilized by physicians to aid in bedside diagnostics. Additional processes may be performed utilizing the auscultatory device. The present process is intended to provide an exemplary use of the auscultatory device in a novel diagnostic technique enabled by the auscultatory device.

The process begins at step 1901 where an acoustic attenuation signal is generated from the acoustic transducer. Such an acoustic signal may be a pulse signal or a continuous acoustic signal. Additionally, the acoustic signal may be at physiological frequencies or at elevated frequencies to increase resolution and eliminate interference.

The process then proceeds to step 1902 where the chest cavity of the patient is measured for sound waves. In this step, a single acoustic sensor may be used to sense heart sounds and attenuation signals. In such embodiments the acoustic sensor must be able to be responsive across a wide frequency range. In some embodiments, more than one sensor may be utilized, each designed to sense acoustic signals within select frequency ranges. Moreover, at least one of the sensors, in some embodiments, may be the transducer that generates the attenuation signal. In these embodiments, the echo of the generated acoustic signal is sensed.

The process then proceeds to step 1910 where pressure of chest pad application to the patient is measured. This pressure measurement may be made by the pressure sensor as illustrated in any of the exemplary illustrations of FIGS. 27A to 29. Chest pad application is inherently variable, and a pressure sensor may be utilized to aid in signal calibration by accounting for this variability in pad application. It should be noted, however, that this calibration is optional. Thus, in some embodiments, step 1910 may be omitted in systems that are not enabled to measure chest pad application pressure.

The process then proceeds to step 1904 where an inquiry is made if the acoustic signals are received on a single channel. If the acoustic signals are on a single channel, which is the case where a single acoustic sensor is used to sense heart sounds and attenuation signals, then the process proceeds to step 1903 where the acoustic signals are filtered by frequency. High frequency attenuation signals are thus separated from the low frequency heart sounds. The process then proceeds to step 1905, where signal processing is performed.

If at step 1904 separate channels are utilized for heart sound signals and attenuation signals, then the process may proceed directly to the signal processing of step 1905.

The process then proceeds to step 1906 where intensity ratios are generated. The intensity ratio is the acoustic intensity of S1 divided by the attenuation measures Sc. Additionally, the intensity ratio may be further calibrated by utilizing the pressure of the chest pad against the patient's body, as measured at step 1910.

Lastly the process proceeds to step 1907 where cardiac contractility may be determined. By using the intensity ratio (S1/Sc), and the ratio between the 2 main heart sounds (S1/S2), the current cardiac contractility may be estimated. The cardiac contractility may comprise ejection fraction and/or rate of change in pressure in the heart. The process then ends.

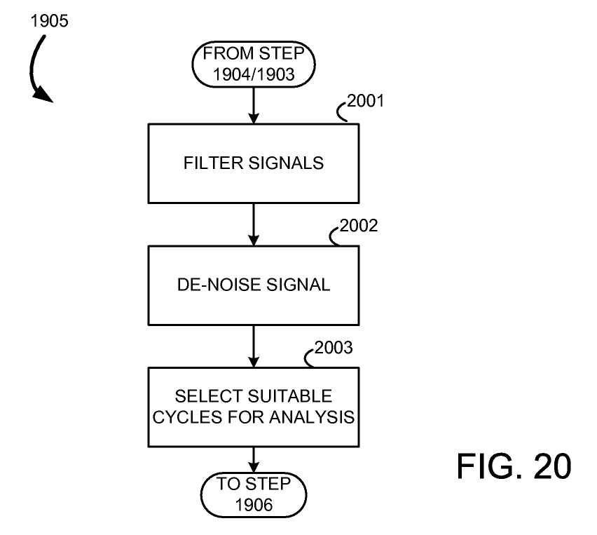

FIG. 20 shows an exemplary process for signal processing for cardiac contractility determination, shown generally at step 1905. The process begins from step 1904 or step 1903. Then at step 2001, signals may be filtered. The process then proceeds to step 2002 where signals are de-noised. Then at step 2003 suitable cycles may be selected for analysis. In some embodiments, each patient has recordings from 3 different sites for extended durations, as well as an ECG recording. A 20 second sound recording may result in a number of heart sound cycles per site depending on the patient's heart rate. On some patients almost all of the cycles may be usable except for occasional spikes present in data. On others, there will be 2 or 3 useful cycles because of noise. In some embodiments, one method for cycle selection is to choose the median of the data. For example, all S1 and S2 amplitudes for a patient at the Pulmonic location are found. The median S1 amplitude as the representative S1 and the median S2 as the representative S2 (Note that these may not occur during the same cycle) may be selected, and then the median Signal to Noise Ratio (SNR) of all the cycles may be generated and used as the general indicator of the SNR of the entire recording. Alternate cycle selection may be utilized such as discarding all cycles below a given SNR level, use the mean of the amplitudes instead of the median, selection of the ‘best’ cycle in an entire recording (such as highest SNR) and use only the S1 and S2 from that cycle, selecting a cycle depending on a specific part of the breathing cycle, or any other appropriate cycle selection method. The process then ends by proceeding to step 1906.

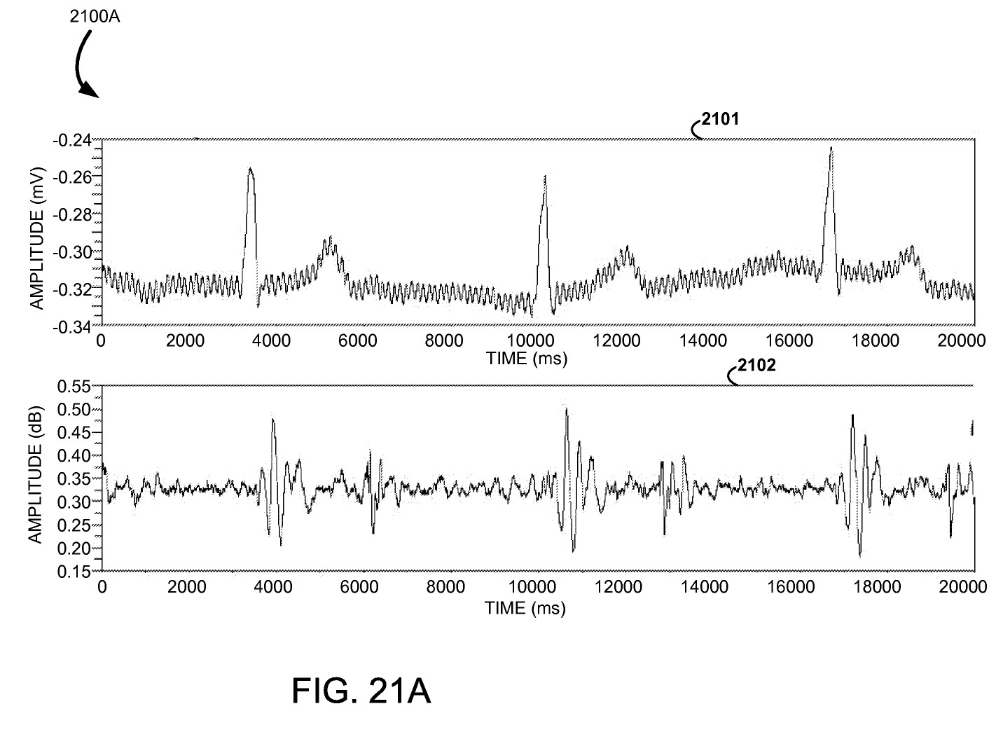

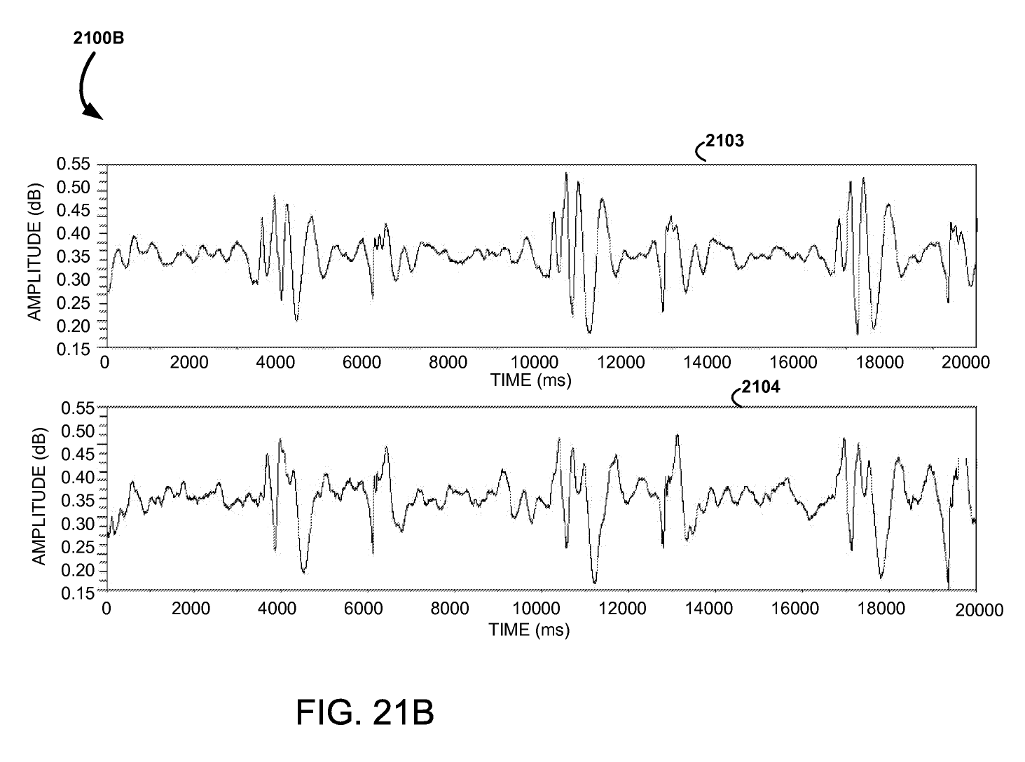

FIG. 21A shows an exemplary illustration of ECG and sound wave measurements for usage by cardiac contractility analysis, shown generally at 2100A. The first plot 2101 is the ECG capture, and the subsequent plot is from a microphone at the Pulmonic location 2102. FIG. 21B shows an exemplary illustration of sound wave measurements for usage by cardiac contractility analysis, shown generally at 2100B. These plots are from microphones at the Apex and Aortic locations, 2103 and 2104, respectively. Each plotting is graphed along a linear timescale. S1 is seen clearly in each plot shortly after the QRS peak in the ECG, and S2 appears shortly after the T wave.

Using the exemplary data, all QRS points in the ECG data are found, which marks the beginning of each heart cycle. Since two adjacent QRS points demarcate one cycle, in the first third of that cycle, looking for the maximum and minimum signal amplitude delineates the S1 signal. The difference of maximum and minimum signal amplitude is S1 amplitude. In the next third of the cycle look once again for the maximum and minimum, the difference of which is the S2 amplitude.

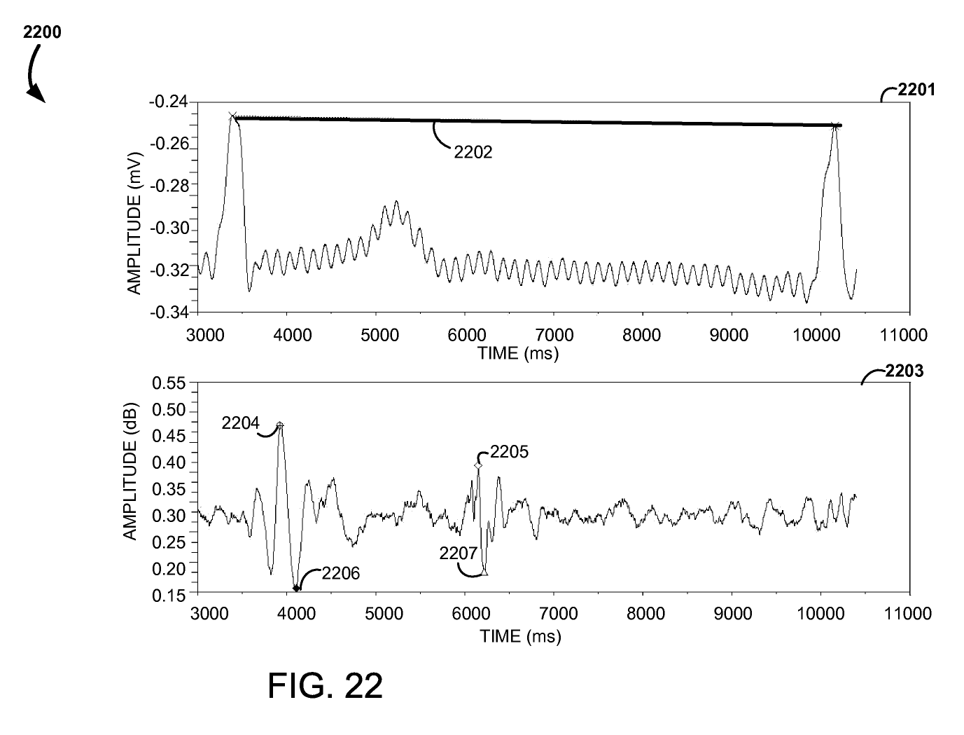

FIG. 22 shows an exemplary illustration of isolated ECG and sound wave measurement for usage by cardiac contractility analysis, shown generally at 2200. Two Xs mark the two subsequent QRSs in the subject's ECG 2201, and there is a Line 2202 between them.

In the heart sound graph 2203, during the first third of that interval, the minimum and maximum is found 2206 and 2204, respectively. During the next third of that cycle, the minimum and maximum is found 2207 and 2205, respectively. The difference between the minima and the maxima are the amplitudes of the S1 and S2, respectively.

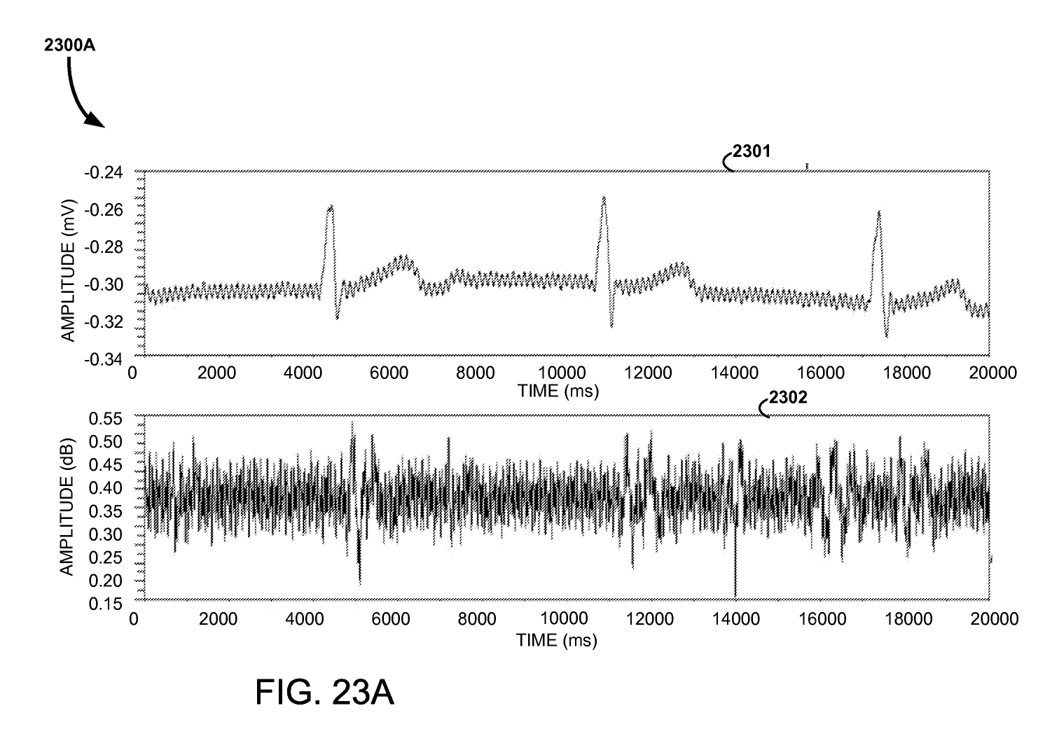

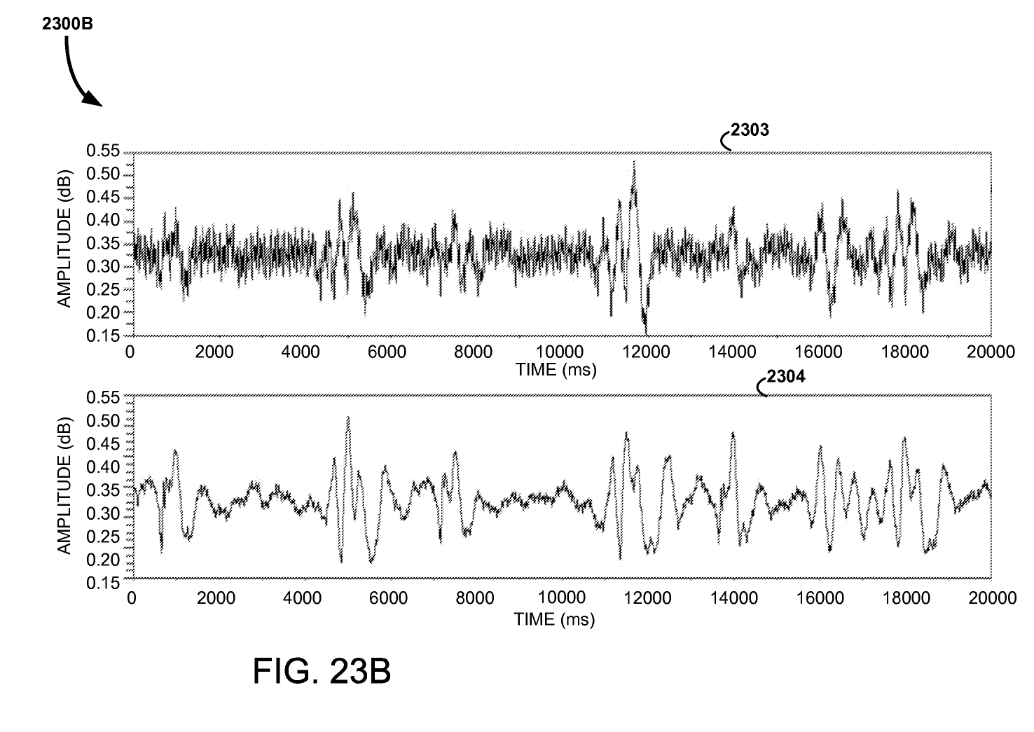

FIG. 23A shows an exemplary illustration of ECG and sound wave measurements when attenuation signal is used for cardiac contractility analysis, shown generally at 2300A. The first plot 2301 is the ECG capture, and the subsequent plot is from the microphone at the Pulmonic 2302. FIG. 23B shows an exemplary illustration of sound wave measurements when attenuation signal is used for cardiac contractility analysis, shown generally at 2300B. The plots are from the microphones at the Apex and Aortic locations 2303 and 2304, respectively. Each plotting is graphed along a linear timescale.

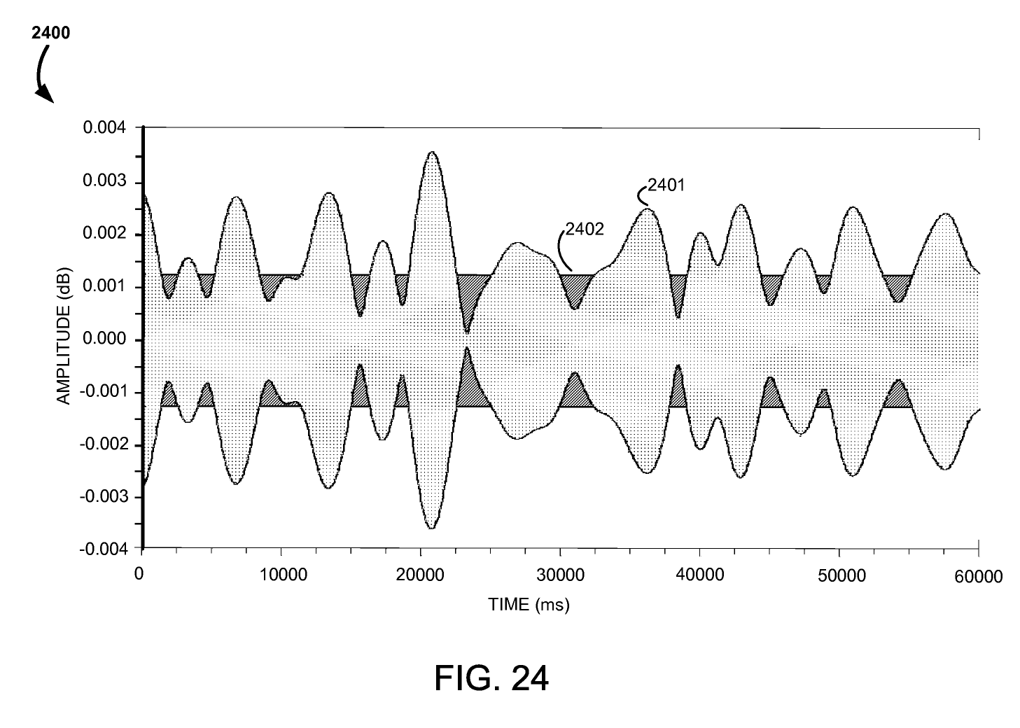

FIG. 24 shows an exemplary illustration of filtered measured transduction signals for cardiac contractility analysis, shown generally at 2400. The data collected during the operation of the transducer is passed through a very narrow band pass filter for each tone, and the Amplitude 2401 of the output from the filter is taken as the amplitude of the tone at that location. In some embodiments, the amplitude of the tone has been defined as 4 times the Standard Deviation 2402 after the narrowband filter. Even at the narrow frequency range, the amplitude of the data fluctuates with breathing cycles and additive/subtractive effects of noise and other data within that band.

In some embodiments, the filter is a bandpass filter with appropriate cutoffs.

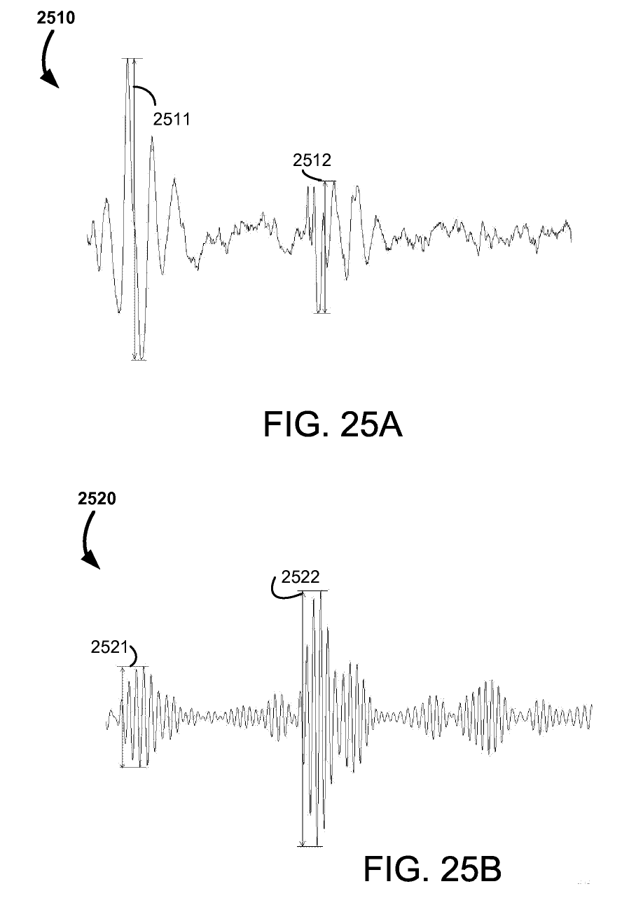

FIG. 25A shows an exemplary illustration of pre-filtered measured heart signals for cardiac contractility analysis, shown generally at 2510. 2511 and 2512 represent the total intensity of the first and second heart sound respectively. FIG. 25B shows an exemplary illustration of post-filtered measured heart signals for cardiac contractility analysis, shown generally at 2520. 2521 and 2522 represent the total intensity of the first and second heart sound, respectively, after the filtering operation.

Certain combinations of these intensities correlate well with particular pathologies. One such relationship is the ratio of the first heart sound after the filtering operation 2521 divided by the unfiltered first heart sound 2511 vs. cardiac contractility when the filtering operation has been a band pass operation centered on a particular frequency band. Another such relationship has been the ratio of the unfiltered first heart sound 2511 divided by the unfiltered second heart sound 2512 vs. cardiac contractility.

It is quite conceivable that there are other relationships between the mentioned intensities 2511, 2512, 2521, 2522 and other cardiac measures. Some of these ratios may also correlate well with the presence of certain cardiac pathologies. For example, after a particular filtering operation, the value of a particular ratio such as the filtering operation 2521 divided by the unfiltered first heart sound 2511 may indicate the presence of a particular cardiac disease such as Aortic Stenosis or Mitral Regurgitation.

Some of these relationships may involve looking at multiple ratios after multiple filtering operations, and a particular pathology might have a distinct frequency signature, whereby looking at a number of ratios over a number of filtering operations might indicate the subject's pathology. A relationship can also be derived by observing the variation in one of the mentioned intensities 2511, 2512, 2521, 2522 over the short or long term.

The filtering operations may involve simple filters, for example a straightforward analog nth order bandpass/lowpass/highpass filter. It is conceivable that wavelet operations, which by their nature divide up the signal into various frequency bands, can also be used to carry out measurements on the heart sound signal.

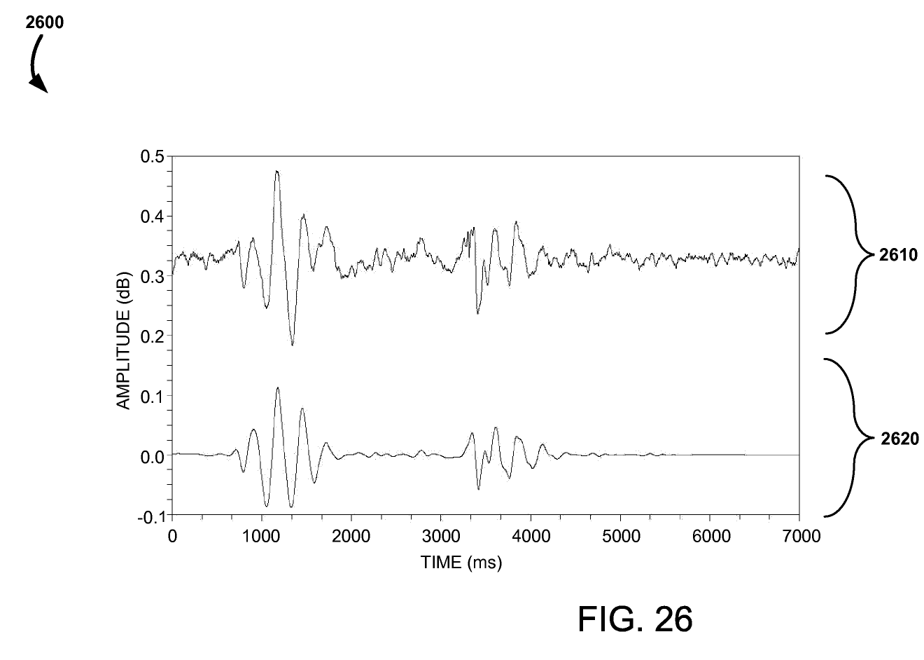

FIG. 26 shows an exemplary illustration of measured heart signals before and after de-noising for cardiac contractility analysis, shown generally at 2600. Issues of noisy recording may affect the intensity of the heart sound parameters being analyzed. This issue may, in some embodiments, be dealt with on two separate levels. First of all, by developing a measure for the noise level within the signal a threshold may be developed to decide whether a particular heart cycle is clean enough to include in measurements. Secondly, data may be cleansed via de-noising.

Signal to Noise Ratio (SNR) may be determined and utilized in the de-noising process. Measuring the noise level, at least in the context of heart sound study, is to measure the power of the signal in a ‘good’ region compared to the power of the signal in a ‘noise’ region.

In some embodiments, the entire signal is filtered with a bandpass filter in the frequency range of the first and second heart sounds.

Wavelet de-noising works quite effectively in removing Gaussian type noise. The de-noising is done on a cycle-by-cycle basis (as opposed to de-noising the entire capture in one go). This does not have a huge impact, except that the noise cutoff thresholds are chosen on a cycle by cycle basis as opposed to one noise threshold for the entire cycle. The graph illustrated at 2610 is an example of recorded heart sounds before de-noising. The graph illustrated at 2620 is an example of the same recorded heart sounds after de-noising.

One novel aspect of the present invention is that all of the capabilities described may be performed in the background—i.e., the image processing extraction of the valve from the motion trace, distance measurements, signal processing for speed determination, Doppler frequency shift, and blood flow estimation. The physicians, or other users, require no new skills to effectuate the system.

Additionally, display of information may be defined based upon statistical confidence levels to minimize misdiagnosis and provide user recommendations. For example, if the valve responsible for a murmur is not reliably detected, say, over 50% of the cardiac cycle, the sensor/transducer may not be pointing in a stable fashion due to hand motion etc., it may indicate repositioning or provide feedback to the user and likewise indicate when the sensor/transducer is pointing accurately at a valve or provide feedback to maximize the motion trace indicating a look direction that sees maximum travel of the leaflet.

Modifications of the present invention are also possible. For example, it is possible to incorporate noise cancellation capability to the embodiments described above, thereby substantially removing ambient environmental noises from the heart sounds received by the acoustic sensors, e.g., sensors of chest-pieces 500, 600, 700.

### Analysis of Cardiac Contractility through Time Interval Measurement

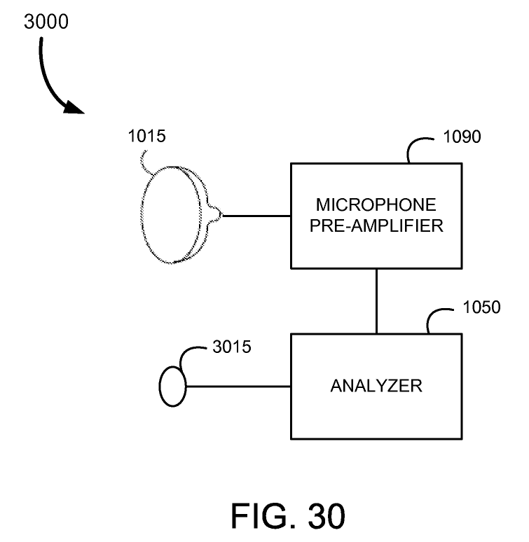

FIG. 30 shows a functional block diagram of another embodiment of the auscultatory device, shown generally at 3000. In some embodiments, the Acoustic Sensor 1015 may be coupled to the Microphone Pre-Amplifier 1090. The Microphone Pre-Amplifier 1090 may in turn couple to the Analyzer 1050. Additionally, an ECG Sensor 3015 may couple to the Analyzer 1050.

In some embodiments, the Acoustic Sensor 1015 may include the functionality of the ECG Sensor 3015. In such embodiments, the ECG Sensor 3015 may be omitted. The purpose of the embodiment shown at 3000 is to illustrate that the determination of rate of pressure change in the heart is not reliant upon attenuation as previously disclosed. In such embodiments, a simple electronic stethoscope and electrocardiogram equipment is required to reliably generate cardiac contractility data for the patient. Cardiac contractility may include the rate of change in pressure in a heart (dP/dt), as well as ejection fraction (EF) of the heart. This enables medical centers to provide a powerful heart diagnostic system with little additional up front costs.

Of course, in some embodiments, a system which includes attenuation as previously disclosed may be utilized to perform the method for generation of cardiac contractility data. Such a system may enable additional calibration of the acoustic data for a more accurate measure of the heart sounds.

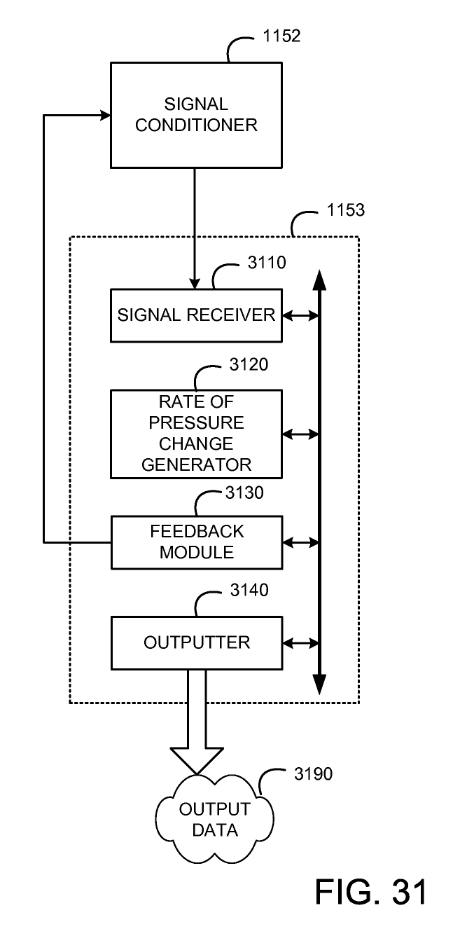

FIG. 31 shows a logical block diagram illustrating an embodiment of 1153. Referring to FIG. 11, Signal Processor 1153 may be seen as an integral component of the Analyzer 1050. The Signal Processor 1153 may, in some embodiments, include a Signal Receiver 3110, a Rate of Pressure Change Generator 3120, a Feedback Module 3130 and an Outputter 3140. Each component may be coupled to each other, thereby enabling, for example, the Rate of Pressure Change Generator 3120 to collect data from the Signal Receiver 3110 and subsequently send results to the Outputter 3140 for outputting.

Raw acoustic and electrocardiography data has been processed by the Signal Conditioner 1152. Processed data is then input to the Signal Receiver 3110. In some embodiments, the Rate of Pressure Change Generator 3120 may analyze the processed data for generation of rates of pressure change in the heart.

The Feedback Module 3130 may also receive processed data from the Signal Receiver 3110, as well as data from the Rate of Pressure Change Generator 3120. Feedback Module 3130 may then provide feedback to the Signal Conditioner 1152.

The Outputter 3140 may receive analyzed data from the Rate of Pressure Change Generator 3120 and output the Output Data 3190 to additional components within the Analyzer 1050.

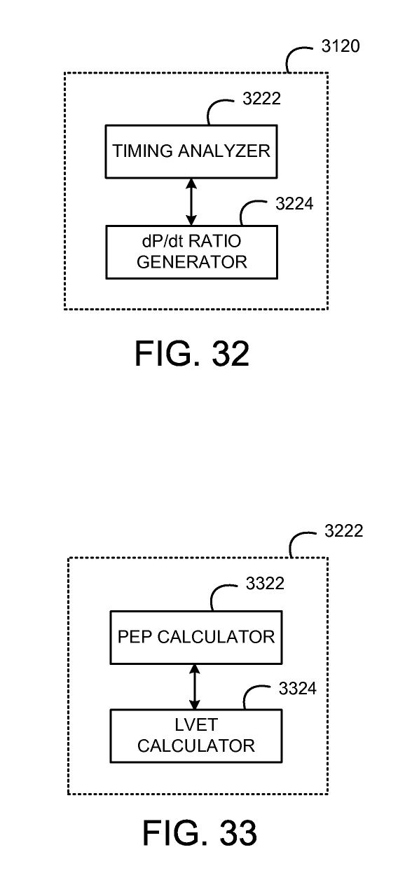

FIG. 32 shows a logical block diagram illustrating an embodiment of the Rate of Pressure Change Generator 3120. The Rate of Pressure Change Generator 3120 may include a Timing Analyzer 3222 and a Max dP/dt Generator 3224. Timing Analyzer 3222 couples to the Max dP/dt Generator 3224.

The Timing Analyzer 3222 may analyze the processed data from the Signal Receiver 3110 and determine timing of S1 and S2 peaks from the acoustic data. The timing data may then be received by the Max dP/dt Generator 3224 for further analysis. The Max dP/dt Generator 3224 may then generate the dP/dt ratio from the timing data.

Of course, additional components may be included within the Rate of Pressure Change Generator 3120 as is desired for increased functionality.

FIG. 33 shows a logical block diagram illustrating an embodiment of the Timing Analyzer 3222. The Timing Analyzer 3222 may include a PEP Calculator 3322 and a LVET Calculator 3324. The PEP Calculator 3322 may couple to the LVET Calculator 3324.

The PEP Calculator 3322 may calculate the Pre-ejection Period (PEP) for the heart patent. The LVET Calculator 3324 may calculate Left Ventricular Ejection Time (LVET) for the heart patient. These timing indices may be provided to the Max dP/dt Generator 3224 for further analysis.

Of course, additional components may be included within the Timing Analyzer 3222 as is desired for increased functionality.

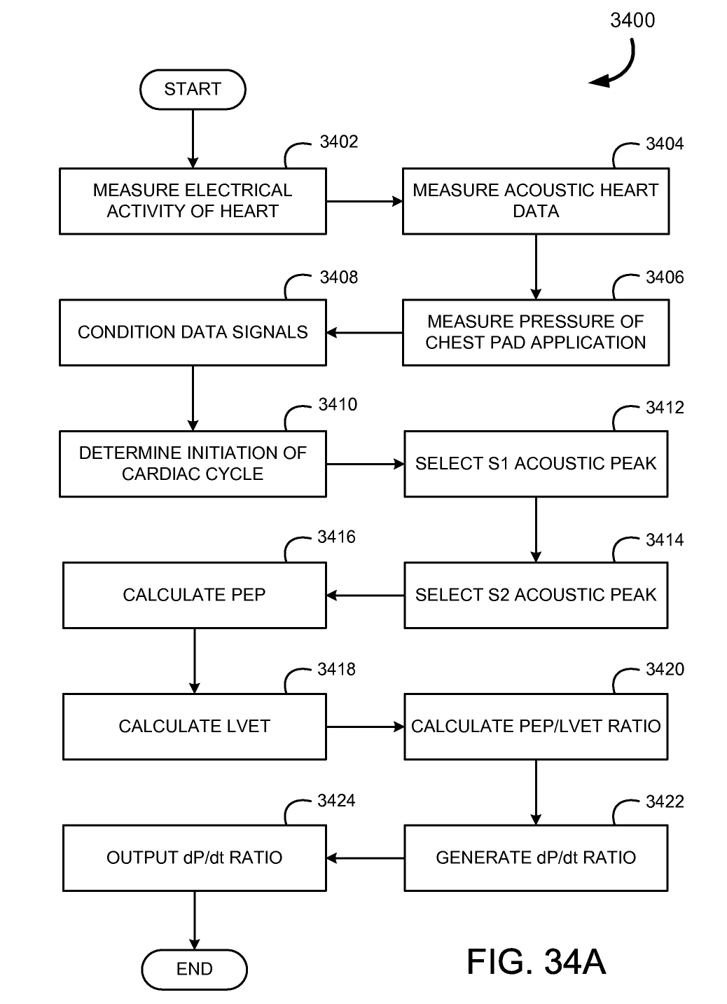

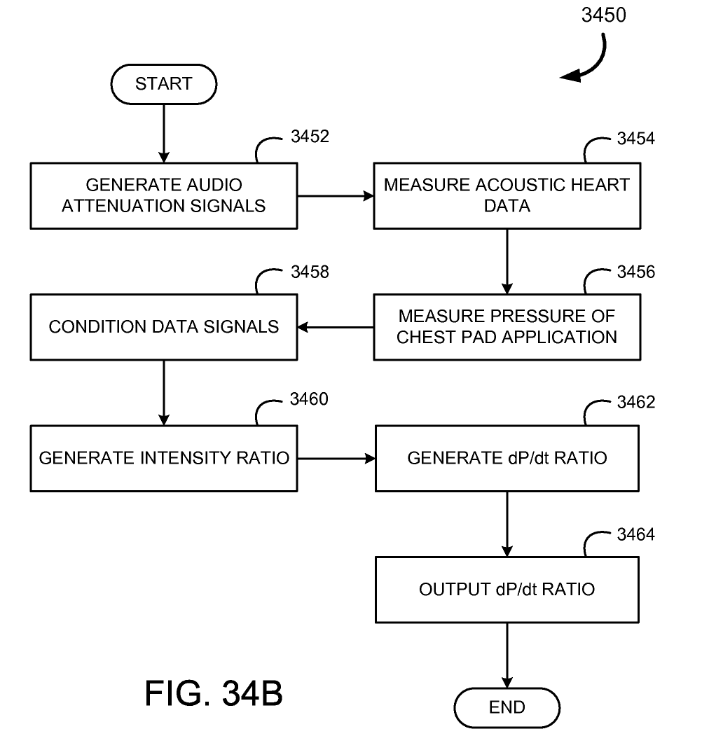

FIG. 34A shows an exemplary process for determining the cardiac contractility in a heart, shown generally at 3400. The process begins at step 3402 where heart electrical activity is measured. This measurement is typically preformed by an Electrocardiogram (ECG or EKG) device. The process then proceeds to step 3404 where acoustic heart sounds are measured. An electronic stethoscope similar to those previously disclosed may be utilized for the measurement of acoustic sounds. In the embodiments where an attenuation enabled system is utilized, attenuation signals may also be recorded at this step.

The process then proceeds to step 3406 where pressure of chest pad application to the patient is measured. This pressure measurement may be made by the pressure sensor as illustrated in any of the exemplary illustrations of FIGS. 27A to 29. Chest pad application is inherently variable, and a pressure sensor may be utilized to aid in signal calibration by accounting for this variability in pad application. It should be noted, however, that this calibration is optional. Thus, in some embodiments, step 3406 may be omitted in systems that are not enabled to measure chest pad application pressure.

The process then proceeds to step 3408 where data signals are conditioned. This may take place within the Signal Conditioner 1152. Then, at step 3410, initiation of the cardiac cycles is determined. This determination may utilize the electrocardiograph data. The process then proceeds to step 3412 where the acoustic peak of the first heart sound (S1) is selected. Then, at step 3414, the acoustic peak of the second heart sound (S2) is selected.

Results from step 3410 and 3412 may be utilized at step 3416 where Pre-ejection Period (PEP) is calculated. Then results from step 3412, 3414 and 3416 may be utilized at step 3418 where Left Ventricular Ejection Time (LVET) is calculated.

The process then proceeds to step 3420 where PEP/LVET ratio is calculated. Results from step 3416 and 3418 are utilized in generation of the PEP/LVET ratio. The process then proceeds to step 3422 where cardiac contractility is generated. The cardiac contractility may be found by correlation to PEP/LVET ratio as well as correlation to PEP value. In addition, the amplitudes of the first and second heart sounds can also be utilized to estimate cardiac contractility.

The process then proceeds to step 3424 where results of the generation of cardiac contractility are output. The process then concludes.

FIG. 34B shows an alternate exemplary process for determining the cardiac contractility in a heart, shown generally at 3450. Such a process may be utilized by physicians to aid in bedside diagnostics. Additional processes may be performed utilizing the auscultatory device. The present process is intended to provide an exemplary use of the auscultatory device in a novel diagnostic technique enabled by the auscultatory device which relies upon acoustic amplitude to generate pressure change in the heart.

The process begins at step 3452 where an acoustic attenuation signal is generated from the acoustic transducer. Such an acoustic signal may be a pulse signal or a continuous acoustic signal. Additionally the acoustic signal may be at physiological frequencies or at elevated frequencies to increase resolution and eliminate interference.

The process then proceeds to step 3454 where the chest cavity of the patient is measured for sound waves. In this step, a single acoustic sensor may be used to sense heart sounds and attenuation signals. In such embodiments, the acoustic sensor must be able to be responsive across a wide frequency range. In some embodiments, more than one sensor may be utilized, each designed to sense acoustic signals within select frequency ranges. Moreover, at least one of the sensors, in some embodiments, may be the transducer that generates the attenuation signal. In these embodiments, the echo of the generated acoustic signal is sensed.

The process then proceeds to step 3456 where pressure of chest pad application to the patient is measured. This pressure measurement may be made by the pressure sensor as illustrated in any of the exemplary illustrations of FIGS. 27A to 29. Chest pad application is inherently variable, and a pressure sensor may be utilized to aid in signal calibration by accounting for this variability in pad application. It should be noted, however, that this calibration is optional. Thus, in some embodiments, step 3456 may be omitted in systems that are not enabled to measure chest pad application pressure.

The process then proceeds to step 3458, where signal conditioning is performed. The process then proceeds to step 3460 where intensity ratios are generated. The intensity ratio is the acoustic intensity of S1 divided by the attenuation measures Sc. Additionally, the intensity ratio may be further calibrated by utilizing the pressure of the chest pad against the patient's body, as measured at step 3456.

The process then proceeds to step 3462 where cardiac contractility is generated. The cardiac contractility may be found by correlation to the generated intensity ratios, as well as to the amplitudes of the first and second heart sounds. In addition, the timing of the PEP and LVET may be utilized to estimate cardiac contractility.

The process then proceeds to step 3464 where results of the generation of cardiac contractility is output. The process then concludes.

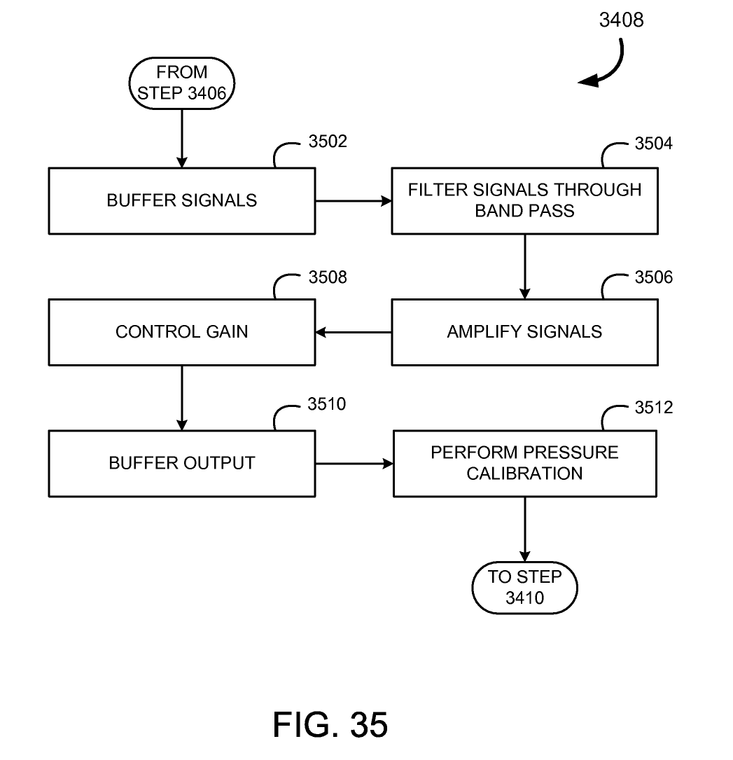

FIG. 35 shows an exemplary process for conditioning data signals, shown generally at 3408. Signal conditioning may occur at the Signal Conditioner 1152.

The process begins from step 3406 from FIG. 34A. The process then proceeds to step 3502 where the input signal is buffered. Buffering occurs at the Input Buffer 1201. Then, at step 3504, the signal may undergo additional filtering. The filtering operations may involve simple filters, for example a straightforward analog Butterworth nth order bandpass/lowpass/highpass filter. It is conceivable that wavelet operations, which by their nature divide up the signal into various frequency bands, can also be used to carry out measurements on the heart sound signal. Additional filtering techniques may be employed as is known by those skilled in the art. Filtering may occur at the Band Pass Filter(s) 1202.

The process then proceeds to step 3506 where the filtered signal is amplified. The process then proceeds to step 3508 where gain may be automatically controlled. A Variable Gain Amplifier 1203 in conjunction with the Gain Controller 1204 may effectuate automatic gain control.

The process then proceeds to step 3510 where the output is buffered. The Output Buffer 1205 may perform this operation. Then, at step 3512, pressure calibration may be performed.

Pressure calibration may be an optional step. When pressure of chest pad application is measured at step 3406 of FIG. 34A then it is appropriate to calibrate for this measurement.

Additional signal conditioning steps may be performed as is known by those skilled in the art. Particularly, additional calibration steps may be performed as is desired to enhance signal accuracy. For example, calibration for acoustic attenuation may be desired when utilizing a system capable of determining acoustic attenuation.

The process then ends by proceeding to step 3410.

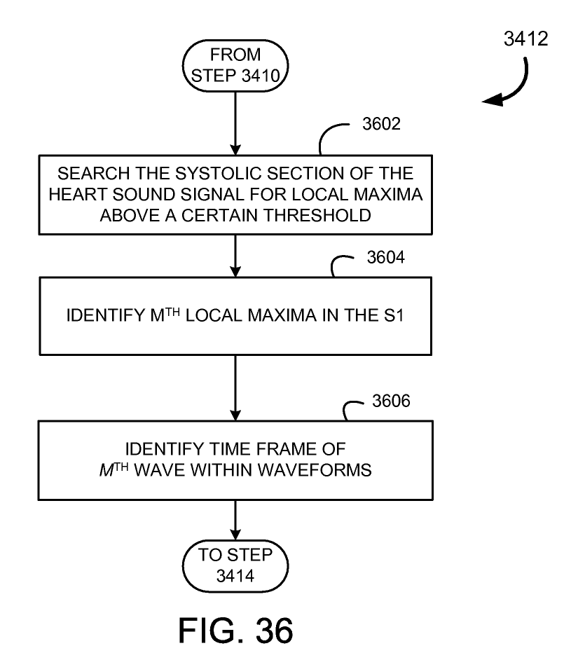

FIG. 36 shows an exemplary process for selecting a S1 acoustic peak, shown generally at 3412. The process begins from step 3410 of FIG. 34A. The process then proceeds to step 3602 where a first acoustic threshold is set. This may be set by the physician, or more typically may be pre calibrated. The purpose of this threshold is to identify waveforms of the recorded acoustic signal that “belong” to the first heart sound (S1). Although the threshold is based on the intensity of the acoustic signal, there will also be a temporal relationship that will need to be satisfied, that is, the S1 is expected to occur in a given time interval.

The process then proceeds to step 3604 where the first consecutive waves, after the initiation of the cardiac cycle, that are above the threshold are identified. Thus, all waveforms of the S1 heart sound that are above the threshold will be identified. Then, at step 3606, the timeframe of the M^{th} wave within the identified set of waveforms is determined. In some embodiments, the M^{th} wave is the first wave of the identified S1 waves. Alternatively, the M^{th} wave may be the second, third or later waveform within identified S1 waves. In some alternate embodiments, the M^{th} wave may be the wave within the identified S1 waves that has the largest amplitude. Decisions as to the definition of the M^{th} wave may be physician defined or prior calibrated. The process then ends by proceeding to step 3414 of FIG. 34A.

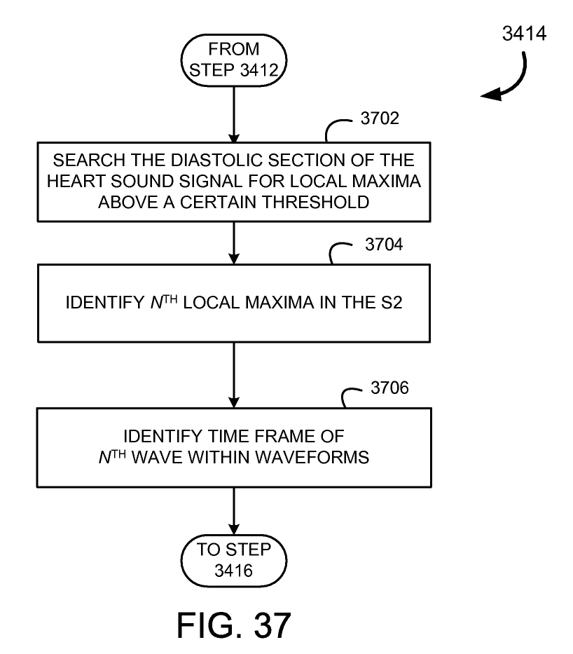

FIG. 37 shows an exemplary process for selecting an S2 acoustic, shown generally at 3414. The process begins from step 3412 of FIG. 34A. The process then proceeds to step 3702 where a second acoustic threshold is set. This may be set by the physician, or more typically may be pre calibrated. The purpose of this threshold is to identify waveforms of the recorded acoustic signal that “belong” to the second heart sound (S2). As with the S1 peaks, the S2 peaks are expected to occur in a given time interval from the initiation of the heart sound cycle.

The process then proceeds to step 3704 where the second consecutive waves, after the initiation of the cardiac cycle, that are above the threshold are identified. Thus, all waveforms of the S2 heart sound that are above the threshold will be identified. Then, at step 3706, the timeframe of the N^{th} wave within the identified set of waveforms is determined. This is preformed by subtracting the time that the cardiac cycle was initiated, as measured from the electrocardiograph, from the time of the N^{th} wave. In some embodiments, the N^{th} wave is the first wave of the identified S2 waves. Alternatively, the N^{th} wave may be the second, third or later waveform within identified S2 waves. In some alternate embodiments, the N^{th} wave may be the wave within the identified S2 waves that has the largest amplitude. Decisions as to the definition of the N^{th} wave may be physician defined or prior calibrated. The process then ends by proceeding to step 3416 of FIG. 34A.

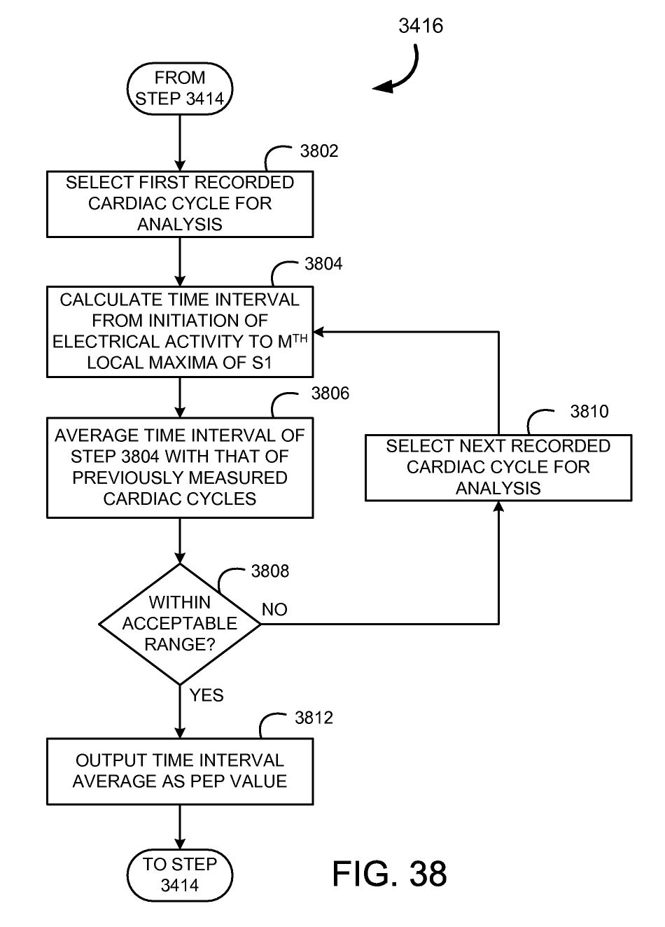

FIG. 38 shows an exemplary process for calculating Pre-ejection Period (PEP), shown generally at 3416. The process begins from step 3414 of FIG. 34A. The process then proceeds to step 3802 where the first recorded heart cycle is selected for analysis. Then, at step 3804 the time that the cardiac cycle was initiated for the analyzed cardiac cycle, as measured from the electrocardiograph, is subtracted from the time of the M^{th} wave for the analyzed cardiac cycle.

The process then proceeds to step 3806 where the time interval calculated at 3804 is averaged with previously calculated time intervals. In the case of the first cardiac cycle, there are no previous cardiac cycles analyzed, therefore the first cycle's time interval becomes the average time interval.

The process then proceeds to step 3808 where an inquiry is then made as to whether the average time interval is within an acceptable range. In some embodiments, determining if the values are acceptable may be a simple inquiry as to whether enough cardiac cycles have been analyzed. In such an embodiment, the number of cardiac cycles that must be analyzed may be pre configured. Alternatively, in some embodiments, the time intervals measured for each cardiac cycle may be compared with one another. In these embodiments, consistency of time intervals may be utilized to determine when the average time interval is acceptable.

If, at step 3808 the average time interval is not acceptable, the process then proceeds to step 3810 where the next cardiac cycle is selected for analysis. The process then proceeds to step 3804 where the time that the cardiac cycle was initiated for the analyzed cardiac cycle, as measured from the electrocardiograph, is subtracted from the time of the M^{th} wave for the analyzed cardiac cycle.

Else, if at step 3808 the average time interval is acceptable, the process then proceeds to step 3812 where the average time interval is output as the Pre-ejection Period (PEP) value. The process then ends by proceeding to step 3418 of FIG. 34A.

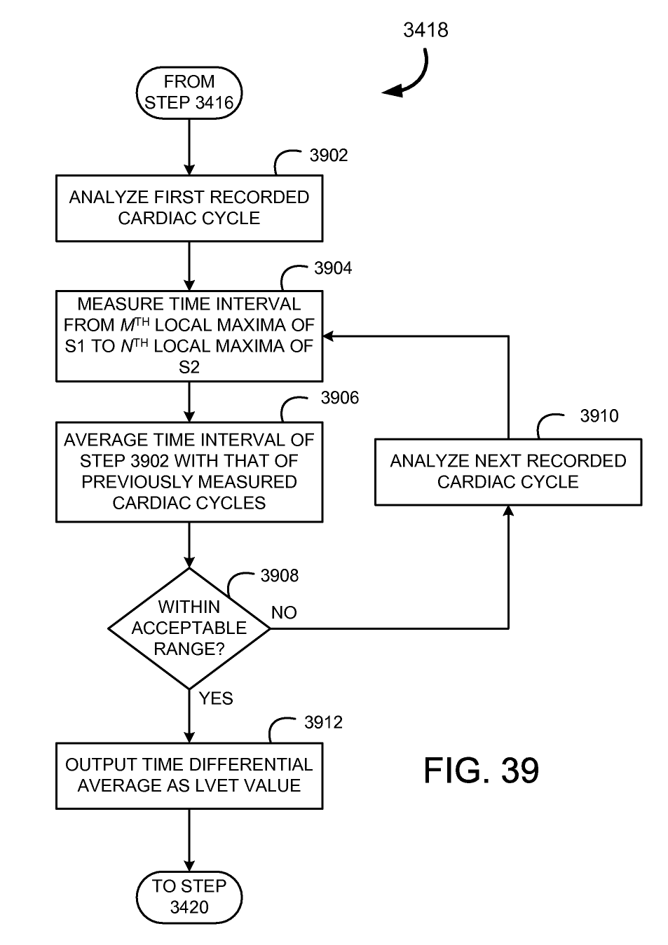

FIG. 39 shows an embodiment of an exemplary process for calculating Left Ventricular Ejection Time (LVET), shown generally at 3418. The process begins from step 3416 of FIG. 34A. The process then proceeds to step 3902 where the first recorded heart cycle is selected for analysis. Then, at step 3904 the time of the M^{th} wave for the analyzed cardiac cycle is subtracted from the time of the N^{th} wave for the analyzed cardiac cycle.

The process then proceeds to step 3906 where the time interval calculated at 3904 is averaged with previously calculated time intervals. In the case of the first cardiac cycle, there are no previous cardiac cycles analyzed, therefore the first cycle's time interval becomes the average time interval.

The process then proceeds to step 3908 where an inquiry is then made as to whether the average time interval is acceptable. In some embodiments, determining if the values are acceptable may be a simple inquiry as to whether enough cardiac cycles have been analyzed. In such an embodiment, the number of cardiac cycles that must be analyzed may be pre configured. Alternatively, in some embodiments the time intervals measured for each cardiac cycle may be compared with one another. In these embodiments, consistency of time intervals may be utilized to determine when the average time interval is acceptable.

If, at step 3908 the average time interval is not acceptable, the process then proceeds to step 3910 where the next cardiac cycle is selected for analysis. The process then proceeds to step 3904 where the time of the M^{th} wave for the analyzed cardiac cycle is subtracted from the time of the N^{th} wave for the analyzed cardiac cycle.

Else, if at step 3908 the average time differential is acceptable, the process then proceeds to step 3912 where the average time differential is output as the LVET value. The process then ends by proceeding to step 3420 of FIG. 34A.

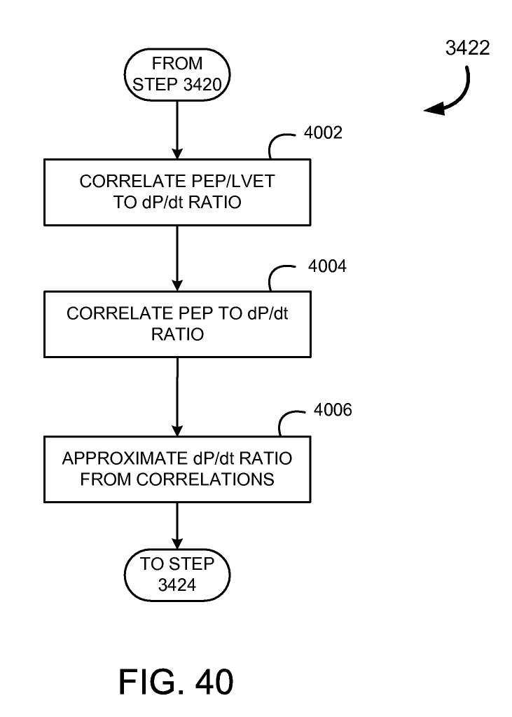

FIG. 40 shows an exemplary process for generating the cardiac contractility in a heart, shown generally at 3422. The process begins from step 3420 of FIG. 34A. The process then proceeds to step 4002 where the ratio of PEP/LVET is correlated to the cardiac contractility. Then, at step 4004 the calculated Pre-ejection Period (PEP) may be correlated to the cardiac contractility. Both PEP/LVET and PEP have a linear relationship to the cardiac contractility for the heart. Thus, at step 4006 the value of the cardiac contractility may be approximated from the correlations. The process then ends by proceeding to step 3424 of FIG. 34A.

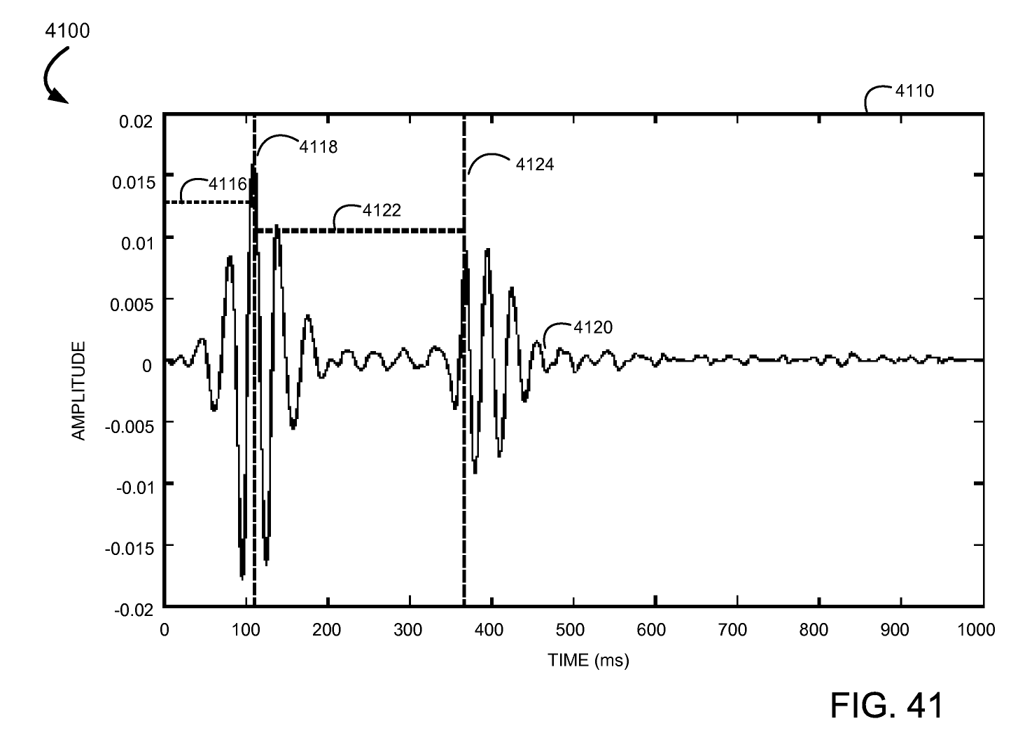

FIG. 41 shows an exemplary illustration of a sound wave measurement for usage in determining the rate of pressure change in a heart, shown generally at 4100. The Heart Sound Plot 4110 is shown with an Amplitude Axis 4102 plotted against a Time Axis 4104. Time Axis 4104 may be measured in milliseconds as an entire cardiac cycle is typically on the order of one second. Amplitude Axis 4102 may be measured in decibels (db), or other appropriate measurement.

The sound waves of the Heart Sound Waveform 4120 may be seen, with S1 and S2 clearly evident. Initiation of the cardiac cycle, as measured by the electrocardiogram (ECG), is at the zero time mark. The M^{th} Wave 4118 may be seen as occurring at roughly 110 ms. The Pre-ejection Period 4116 may be seen as the time difference between the M^{th} Wave 4118 and the initiation of the cardiac cycle.

The N^{th} Wave 4124 may be seen as occurring at roughly 380 ms. The Left Ventricular Ejection Time 4122 may be seen as the time difference between the N^{th} Wave 4124 and the M^{th} Wave 4118, or roughly 270 ms. From this data, the PEP/LVET ratio may be calculated and the cardiac contractility may be generated.

In sum, the present invention provides many advantages over many existing heart diagnostic systems, including ease of use, portability, cost effectiveness, and noninvasiveness. The present invention also allows for the powerful generation of cardiac contractility. Cardiac contractility includes the rate of pressure change (dP/dt) and ejection fraction (EF). Cardiac contractility in a heart patient may be determined by combining an acoustic sensing with an ECG sensing. Alternatively, cardiac contractility may be determined through calibrated acoustic measurements.

While this invention has been described in terms of several preferred embodiments, there are alterations, modifications, permutations, and substitute equivalents, which fall within the scope of this invention. Although sub-section titles have been provided to aid in the description of the invention, these titles are merely illustrative and are not intended to limit the scope of the present invention. For example, the techniques described in the measurement of the ejection fraction by looking at calibrated heart sound amplitudes can be equally applied to the determination of dP/dt. A calibrated first heart sound can be used to measure dP/dt either by itself or in combination with the timing interval techniques described in this section.

It should also be noted that there are many alternative ways of implementing the methods and apparatuses of the present invention. It is therefore intended that the following appended claims be interpreted as including all such alterations, modifications, permutations, and substitute equivalents as fall within the true spirit and scope of the present invention.

What is claimed is:

1. A method for cardiac contractility analysis, useful in association with a cardiac patient, and an auscultation device having a transducer, a sensor and a heart sound processor, the method comprising:
orienting the transducer on a first location of the cardiac patient;
orienting the sensor on a second location of the cardiac patient, wherein the sensor includes a pressure sensor;
measuring pressure of the sensor on the second location of the cardiac patient;
generating an audio signal at the first location of the cardiac patient by utilizing the transducer;
receiving an attenuated audio signal resulting from the generated audio signal, wherein the attenuated audio signal is received at the second location of the cardiac patient by the sensor;
receiving a heart sound signal at the second location of the subject by the sensor, wherein the heart sound signal includes a first acoustic peak and a second acoustic peak;
computing an acoustic attenuation between the first location and the second location based on differences between the generated audio signal and the received attenuated audio signal;
computing an intensity ratio by dividing an amplitude of the heart sound signal by the acoustic attenuation;
calibrating the intensity ratio utilizing the measured pressure of the sensor on the second location of the cardiac patient;
calculating amplitude of the first acoustic peak;
calculating amplitude of the second acoustic peak; and
computing the cardiac contractility by correlation to the computed intensity ratio, amplitude of the first acoustic peak and amplitude of the second acoustic peak.

2. A method for cardiac contractility analysis, useful in association with a cardiac patient, and an auscultation device having a sensor and a heart sound processor, the method comprising:
orienting the sensor on the cardiac patient;
receiving a heart sound signal of the cardiac patient by the sensor, wherein the heart sound signal includes a first heart sound and a second heart sound;
calibrating the first heart sound utilizing the second heart sound; and
computing the cardiac contractility by correlation to the calibrated first heart sound.

3. The method of claim 2 further comprising:
measuring pressure of the sensor on the cardiac patient using a pressure sensor, wherein the sensor includes the pressure sensor; and
calibrating the received heart sounds using the pressure measurement.

4. The method of claim 2 further comprising:
measuring electrical activity of the heart;
determining initiation of cardiac cycle using the measured electrical activity;
identifying a first acoustic peak of the first heart sound caused by the closure of atrioventricular valves in the heart;
calculating pre-ejection period of the heart by measuring a first time interval from the initiation of the cardiac cycle to the first acoustic peak; and
verifying the cardiac contractility by correlation to pre-ejection period.

5. The method of claim 4 further comprising:
identifying a second acoustic peak of the second heart sound caused by the closure of semilunar valves in the heart;
calculating left ventricular ejection time of the heart by measuring a second time interval from the first acoustic peak to the second acoustic peak;
calculating a ratio of pre-ejection period over left ventricular ejection time; and
verifying the cardiac contractility by correlation to at least one of the ratio of pre-ejection period over left ventricular ejection time and the pre-ejection period.

6. The method of claim 4 wherein the calculating the cardiac contractility includes averaging pre-ejection period of the heart over a plurality of cardiac cycles.

7. The method of claim 5 wherein calculating the cardiac contractility includes averaging left ventricular ejection time of the heart over a plurality of cardiac cycles.

8. A method for cardiac contractility analysis, useful in association with a cardiac patient, and an auscultation device having a sensor and a heart sound processor, the method comprising:
orienting the sensor on a first location of the cardiac patient;
receiving a heart sound signal at the first location of the cardiac patient by the sensor, wherein the heart sound signal includes a first acoustic peak and a second acoustic peak;
calculating amplitude of the first acoustic peak; and
computing the cardiac contractility by correlation to the amplitude of the first acoustic peak.

9. The method of claim 8 further comprising:
orienting a transducer on a second location of the cardiac patient;
generating an audio signal at the second location of the cardiac patient by utilizing the transducer;
receiving an attenuated audio signal resulting from the generated audio signal, wherein the attenuated audio signal is received at the first location of the cardiac patient by the sensor;
computing an acoustic attenuation between the second location and the first location based on differences between the generated audio signal and the received attenuated audio signal; and
calibrating the amplitude of the first acoustic peak using the acoustic attenuation.

10. The method of claim 8 further comprising:
measuring pressure of the sensor on the first location of the cardiac patient using a pressure sensor, wherein the sensor includes the pressure sensor; and
calibrating the amplitude of the first acoustic peak using the pressure measurement.

11. The method of claim 9 further comprising:
measuring electrical activity of the heart;
determining initiation of cardiac cycle using the measured electrical activity;
calculating pre-ejection period of the heart by measuring a first time interval from the initiation of the cardiac cycle to the first acoustic peak; and
verifying the cardiac contractility by correlation to pre-ejection period.

12. The method of claim 11 further comprising:
calculating left ventricular ejection time of the heart by measuring a second time interval from the first acoustic peak to the second acoustic peak;
calculating a ratio of pre-ejection period over left ventricular ejection time; and
verifying the cardiac contractility by correlation to at least one of the ratio of pre-ejection period over left ventricular ejection time and the pre-ejection period.

13. The method of claim 11 wherein the calculating the cardiac contractility includes averaging pre-ejection period of the heart over a plurality of cardiac cycles.

14. The method of claim 12 wherein calculating the cardiac contractility includes averaging left ventricular ejection time of the heart over a plurality of cardiac cycles.

15. A system for cardiac contractility analysis, useful in association with a cardiac patient, the system comprising:
an electrocardiogram configured to measure electrical activity of the heart;
a transducer configured to collect acoustic data from the heart;
a signal processor configured to determine initiation of the cardiac cycle using the measured electrical activity, and identify a first acoustic peak by analyzing the acoustic data, wherein the first acoustic peak identifies a first heart sound caused by the closure of atrioventricular valves in the heart;
an analyzer configured to calculate pre-ejection period of the heart by subtracting timing of the initiation of the cardiac cycle from timing of the first acoustic peak; and
a ratio generator configured to calculate the cardiac contractility by correlation to pre-ejection period.

16. The system of claim 15 further comprising:
the signal processor configured to identify a second acoustic peak by analyzing the acoustic data, wherein the second acoustic peak identifies a second heart sound caused by the closure of semilunar valves in the heart;
the analyzer configured to calculate left ventricular ejection time of the heart by subtracting timing of the first acoustic peak from timing of the second acoustic peak; and
the ratio generator configured calculate a ratio of pre-ejection period over left ventricular ejection time, and generate the cardiac contractility by correlation to at least one of the ratio of pre-ejection period over left ventricular ejection time and the pre-ejection period.

17. The system of claim 16 wherein the system for calculating the cardiac contractility is configured to average pre-ejection period of the heart over a plurality of cardiac cycles.

18. The system of claim 16 wherein the system for calculating the cardiac contractility is configured to average left ventricular ejection time of the heart over a plurality of cardiac cycles.

19. The system of claim 16 wherein the first acoustic peak is the M^{th} waveform of the acoustic data.

20. The system of claim 16 wherein the second acoustic peak is the N^{th} waveform of the acoustic data.
